(19) United States
(12) Patent Application Publication
(10) Pub. No.: US 2021/0196655 A1
(43) Pub. Date: Jul. 1, 2021

(54) COMPOSITION FOR CROSS TALK BETWEEN ESTROGEN RECEPTORS AND CANNABINOID RECEPTORS

(71) Applicant: SeeCure Taiwan Co., Ltd., Kaohsiung City (TW)

(72) Inventors: Tsung-Tien Kuo, Kaohsiung City (TW); David J. Yang, Sugar Land, TX (US); Wei-Chung Chang, Kaohsiung City (TW); Min-Ching Chung, Kaohsiung City (TW); Chi-Shiang Ke, Kaohsiung City (TW)

(73) Assignee: SeeCure Taiwan Co., Ltd., Kaohsiung City (TW)

(21) Appl. No.: 17/191,713

(22) Filed: Mar. 4, 2021

Related U.S. Application Data
(63) Continuation-in-part of application No. 16/131,045, filed on Sep. 14, 2018.

(51) Int. Cl. A61K 31/138; A61K 47/54; A61P 35/00

(52) U.S. Cl. CPC A61K 31/138; A61P 35/00; A61K 47/547

(57) ABSTRACT

A composition for cross talk between estrogen receptors and cannabinoid receptors including a chelator and a receptor ligand is provided. A method of synthesizing the composition is also provided, and the composition may be further prepared in pharmaceutical formulations or kits for therapy or molecular imaging.

### CROSS REFERENCE TO RELATED APPLICATIONS

This application is a continuation-in-part application of and claims the priority benefit of a prior application Ser. No. 16/131,045, filed on Sep. 14, 2018, now pending. The entirety of each of the above-mentioned patent application is hereby incorporated by reference herein and made a part of this specification.

### BACKGROUND

### Technical Field

The present invention generally relates to a composition, in particular, to a composition for cross talk between estrogen receptors and cannabinoid receptors, a method of synthesizing the same, a kit, and an imaging method and a treatment method using the same.

### Description of Related Art

Currently assessment of disease status relies on computed tomography (CT), magnetic resonance imaging (MRI), x-ray or ultrasound. These modalities provide morphological (size, shape) and anatomical information. In addition to these imaging modalities, the treatment endpoints rely almost exclusively on the analysis of biopsies by molecular and histopathological methods which provide a microscopic picture of the general heterogeneous process. However, these prognostic tools do not provide cellular target information, thus, assessment of the effectiveness of therapy is not at optimal.

The development of radiolabeled biochemical compounds to understand molecular pathways has expanded the use of nuclear molecular imaging studies in drug development. Positron emission tomography (PET) and single photon emission computed tomography (SPECT) use radiopharmaceuticals to image, map, and measure target site activities (e.g. angiogenesis, metabolism, hypoxia, apoptosis and proliferation). PET and SPECT agents are also known as micro-dosing agents because there are no detectable pharmacologic effects. [¹⁸F]Fluorodeoxyglucose (FDG), a gold standard for PET, is complementary to the CT and MRI and allows detection of unsuspected distant metastases. Though PET FDG was concordant with the findings of CT and MRI in diagnosing various tumors, FDG also has a drawback. For instance, a significant amount (>95%) of FDG was concentrated in mitochondria fraction and this resulted in an apparent false-positive lesion between inflammation/infection and tumor recurrence. In addition, FDG could not provide accurate information on the prediction of therapeutic response. Thus, it is amenable to develop a radiopharmaceutical beyond FDG that can provide therapeutic indications.

### SUMMARY

Accordingly, the present invention provides a composition for cross talk between estrogen receptors (ERs) and cannabinoid receptors (CBRs) and a novel method of synthesizing the same. The composition may be further prepared in pharmaceutical formulations or kits for therapy or molecular imaging.

The invention provides a composition for cross talk between estrogen receptors and cannabinoid receptors including a chelator and a receptor ligand.

In an embodiment of the invention, the chelator is a nitrogen containing tetraazacyclic ring.

In an embodiment of the invention, the nitrogen containing tetraazacyclic ring is a cyclam, a cyclen, a cyclam-carboxylic acid, or a cyclen-carboxylic acid.

In an embodiment of the invention, the receptor ligand is an estrogen ligand or an anti-estrogen ligand.

In an embodiment of the invention, the estrogen ligand includes estradiol, estrone, estiol, and clomiphene.

In an embodiment of the invention, the anti-estrogen ligand includes non-steroidal tamoxifen, torimiphene, raloxifen, and aminoglutethimide.

In an embodiment of the invention, the receptor ligand has a spacer hydroxy group.

In an embodiment of the invention, the composition further includes a metal ion.

In an embodiment of the invention, the metal ion is a radionuclide, a non-radioactive metal, or a combination thereof.

In an embodiment of the invention, the radionuclide is ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, ⁴⁵Ti, or a combination thereof.

In an embodiment of the invention, the non-radioactive metal is a technetium ion (Tc), a stannous ion (Sn), a copper ion (Cu), an indium ion (In), a thallium ion (T1), a gallium ion (Ga), an arsenic ion (As), a rhenium ion (Re), a holmium ion (Ho), a yttrium ion (Y), a samarium ion (Sm), a selenium ion (Se), a strontium ion (Sr), a gadolinium ion (Gd), a bismuth ion (Bi), an iron ion (Fe), a manganese ion (Mn), a lutecium ion (Lu), a cobalt ion (Co), a platinum ion (Pt), a calcium ion (Ca), a rhodium ion (Rh), an europium ion (Eu), and a terbium ion (Tb), or a combination thereof.

In an embodiment of the invention, the composition is a ^{99m}Tc-cyclam-tamoxifen analogue or a ^{99m}Tc-cyclen-tamoxifen analogue.

The invention also provides a kit including the composition described above.

The invention further provides a method of synthesizing the composition described above.

In an embodiment of the invention, the receptor ligand is conjugated to a tetracyclic ring with an epoxide.

In an embodiment of the invention, the epoxide is attached to an aliphatic chain of the receptor ligand.

The invention further provides an imaging method for cancer, rheumatoid arthritis, osteoporosis, atherosclerosis, or endometrial tissue including administration of the composition described above.

In an embodiment of the invention, an image is a gamma image, a PET image, a PET/CT image, a SPECT image, a SPECT/CT image, a PET/MRI image, a SPECT/MRI image, or a hybrid image.

In an embodiment of the invention, an imaging dose is defined as a kit.

The invention further provides a treatment method for cancer, rheumatoid arthritis, osteoporosis, atherosclerosis, or endometrial tissue including administration of the composition described above.

Based on the above, the present invention provides the composition for cross talk between the estrogen receptors and the cannabinoid receptors. The hydroxy group is incorporated in the finished product. In the composition of the present invention, the protected chelator is used as to react the expoxylated receptor ligand to form the chelator-receptor ligand conjugate. The technology platform may exploit conjugating antagonists and agonists and seeing their effects in various forms of diseases. Also, the composition may be further prepared in pharmaceutical formulations and kits using chemical procedures known to skilled artisans. In addition, the method of synthesizing the composition is also provided, and the synthesis method may obviate the need of adding protecting groups to the receptor ligand and increase process efficiency and purify of the final product. Besides, the composition of the present invention may be used for imaging or treating estrogen receptors and cannabinoid receptors associated diseases.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A shows the ¹H-NMR spectrum of Compound 1 synthesized in Example 1 of the invention.

FIG. 1B shows the ¹H-NMR spectrum of Compound 2 synthesized in Example 1 of the invention.

FIG. 1C shows the ¹H-NMR spectrum of Compound 3 synthesized in Example 1 of the invention.

FIG. 1D shows the ¹H-NMR spectrum of Compound SC-05-K-1 synthesized in Example 1 of the invention.

FIG. 1E shows the ¹³C-NMR spectrum of Compound SC-05-K-1 synthesized in Example 1 of the invention.

FIG. 1F shows the ¹H-,¹H COSY NMR spectrum of Compound SC-05-K-1 synthesized in Example 1 of the invention.

FIG. 1G shows the ¹H-,¹³C HSQC NMR spectrum of Compound SC-05-K-1 synthesized in Example 1 of the invention.

FIG. 1H shows the ¹H-,¹³C HMBC NMR spectrum of Compound SC-05-K-1 synthesized in Example 1 of the invention.

FIG. 1I shows the LC-MS spectrum of Compound SC-05-K-1 synthesized in Example 1 of the invention.

FIG. 1J shows the HPLC spectrum of Compound SC-05-K-1 synthesized in Example 1 of the invention.

FIG. 1K and FIG. 1L show the radiochemical purity of Composition ^{99m}Tc-SC-05-K-1 synthesized in Example 2 of the invention in two different systems.

FIG. 1M shows the labeling efficiency of Composition ^{99m}Tc-SC-05-K-1 synthesized in Example 2 of the invention.

FIG. 2A shows the ¹H-NMR spectrum of Compound 5 synthesized in Example 3 of the invention.

FIG. 2B shows the ¹H-NMR spectrum of Compound 6 synthesized in Example 3 of the invention.

FIG. 2C shows the ¹H-NMR spectrum of Compound SC-05-L-1 synthesized in Example 3 of the invention.

FIG. 2D shows the ¹³C-NMR spectrum of Compound SC-05-L-1 synthesized in Example 3 of the invention.

FIG. 2E shows the ¹H-,¹H COSY NMR spectrum of Compound SC-05-L-1 synthesized in Example 3 of the invention.

FIG. 2F shows the ¹H-,¹³C HSQC NMR spectrum of Compound SC-05-L-1 synthesized in Example 3 of the invention.

FIG. 2G shows the ¹H-,¹³C HMBC NMR spectrum of Compound SC-05-L-1 synthesized in Example 3 of the invention.

FIG. 2H shows the LC-MS spectrum of Compound SC-05-L-1 synthesized in Example 3 of the invention.

FIG. 21 shows the HPLC spectrum of Compound SC-05-L-1 synthesized in Example 3 of the invention.

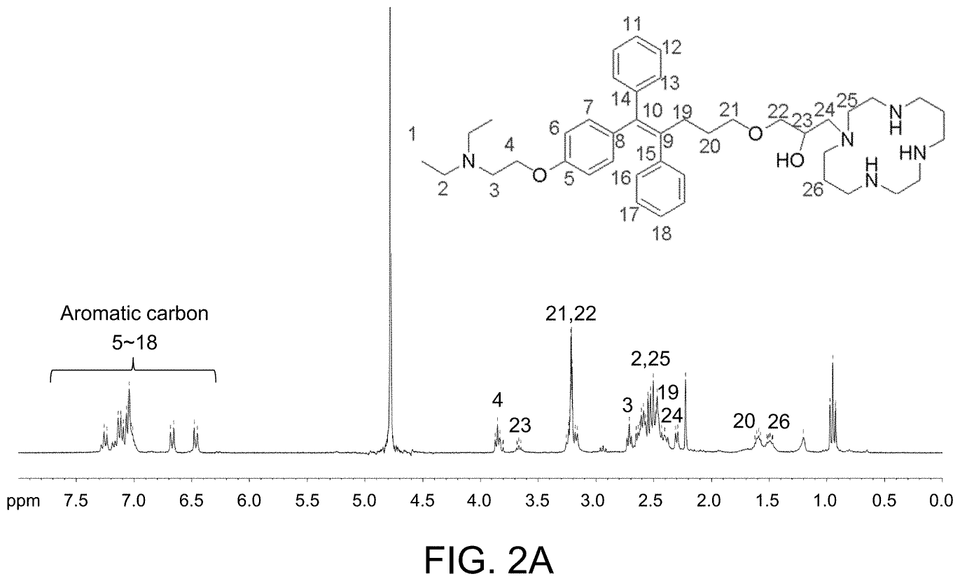

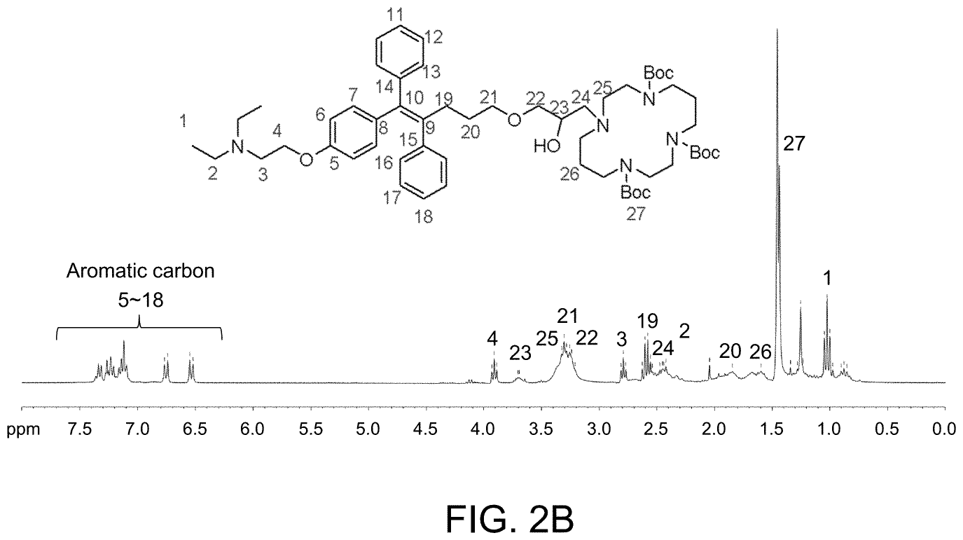

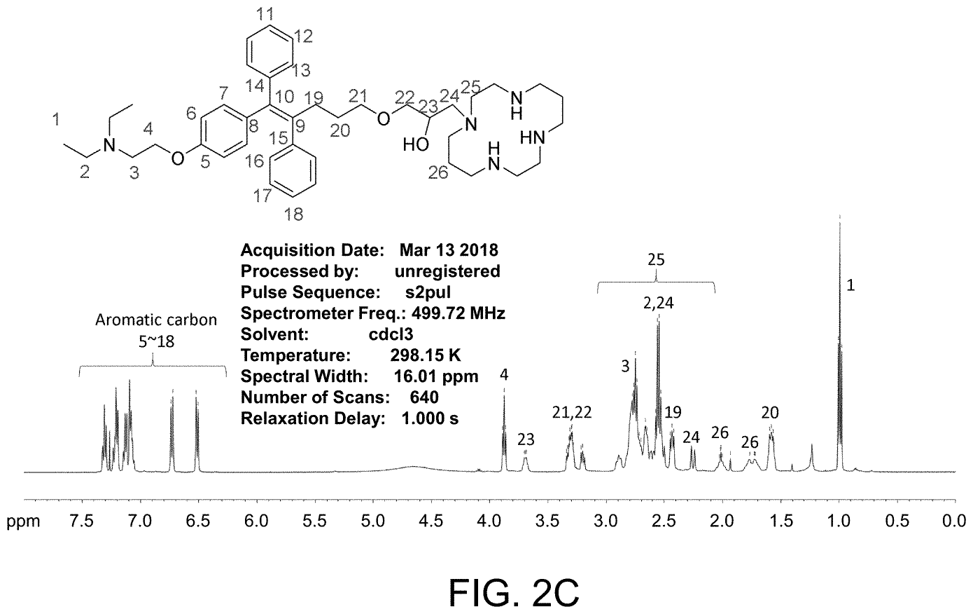

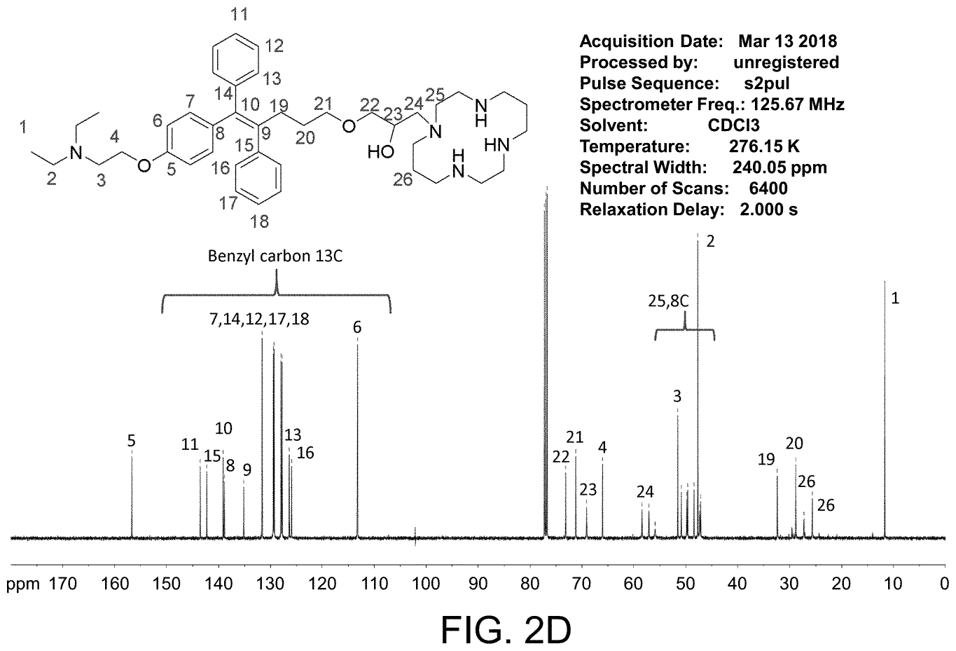

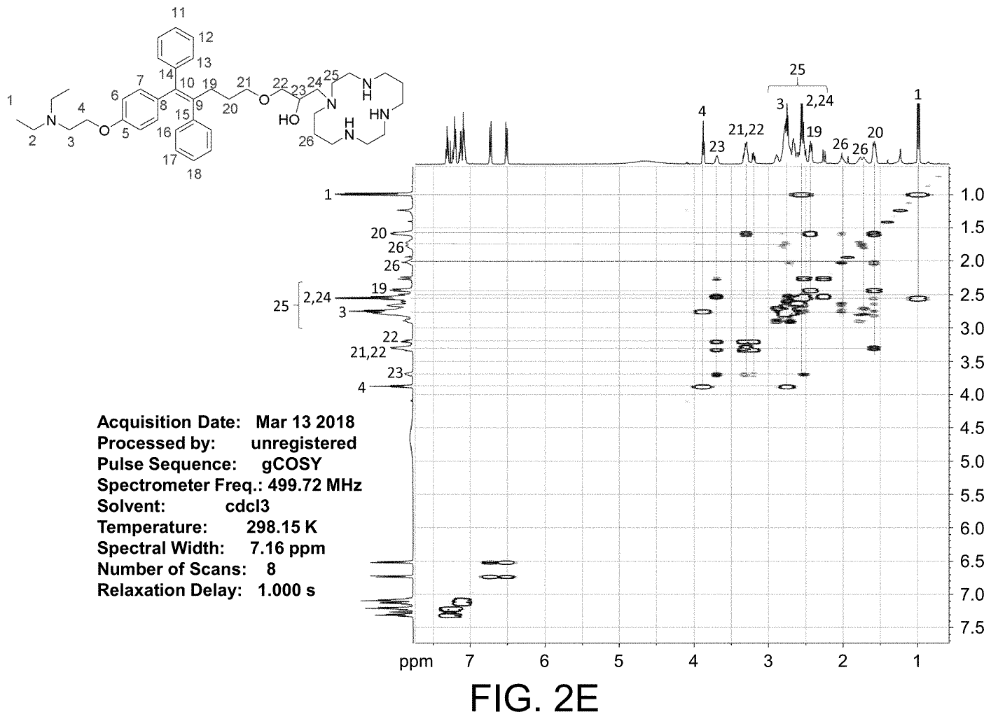

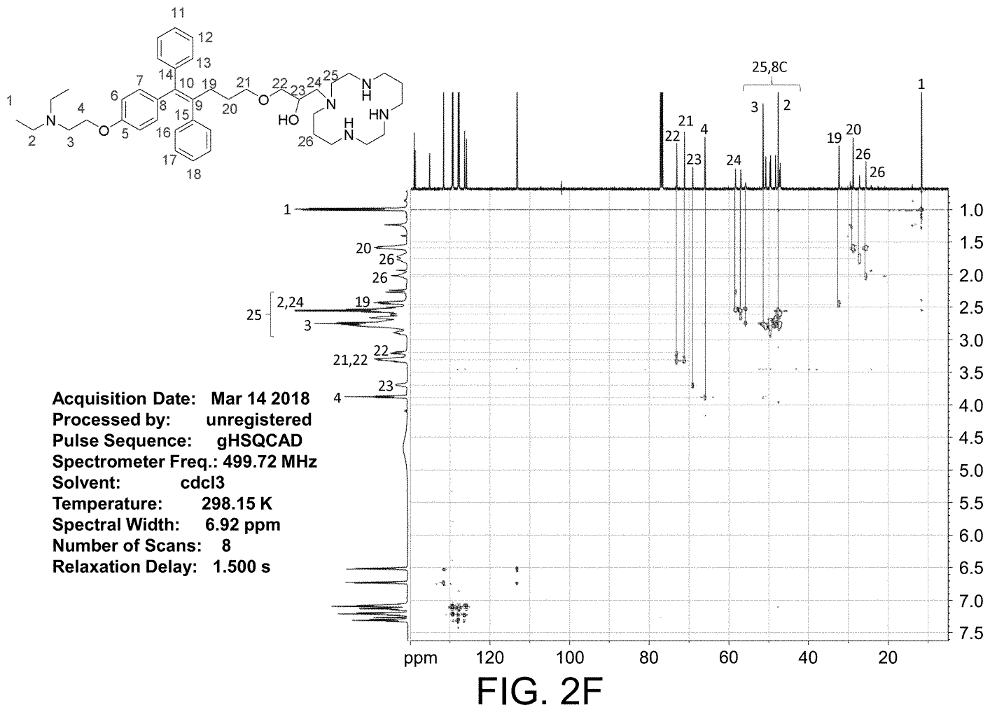

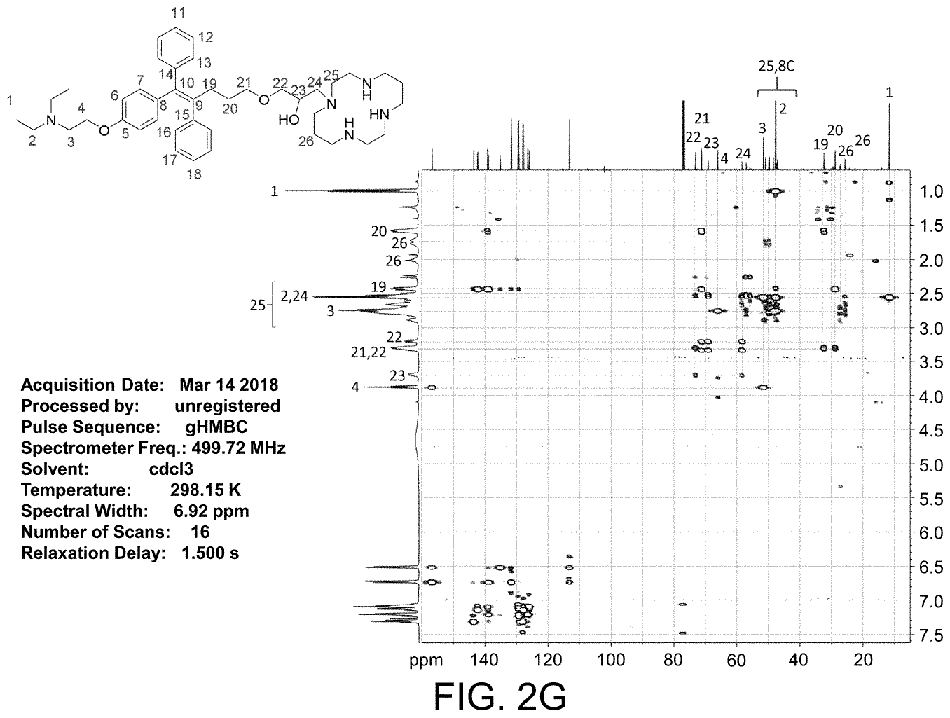

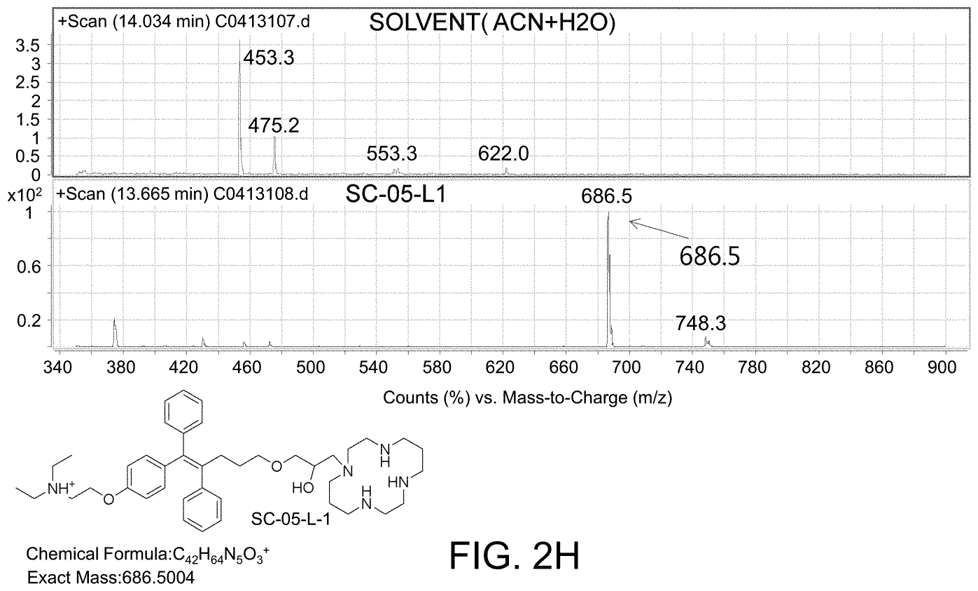

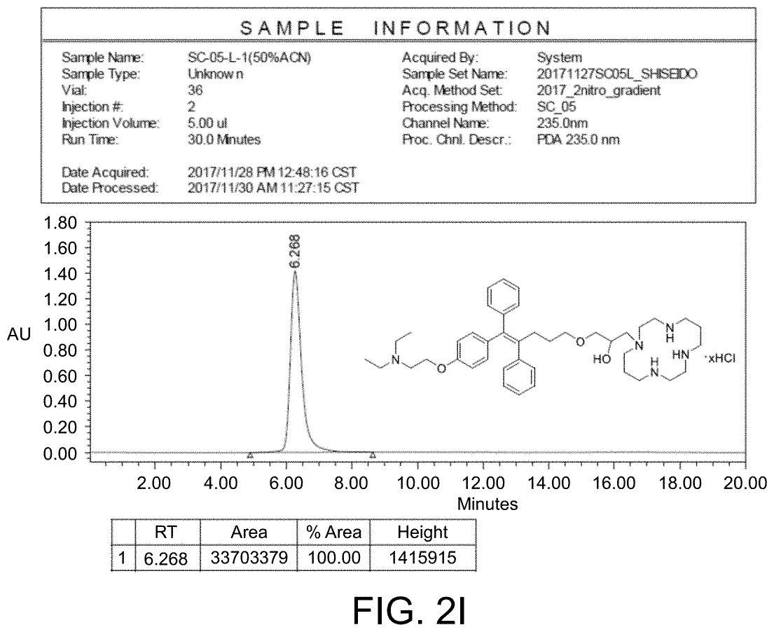

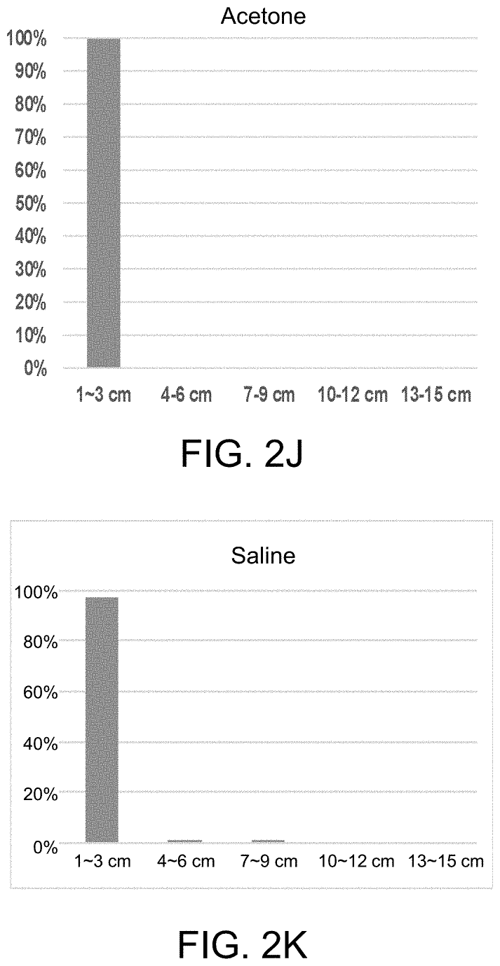

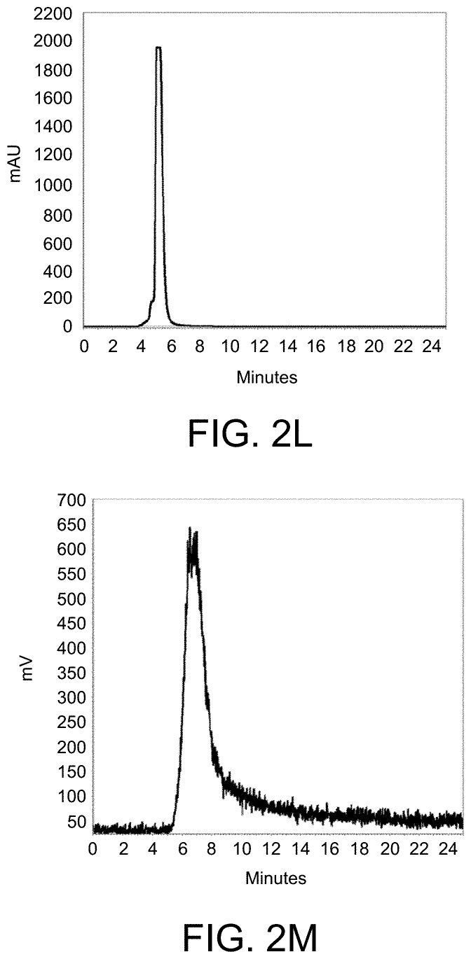

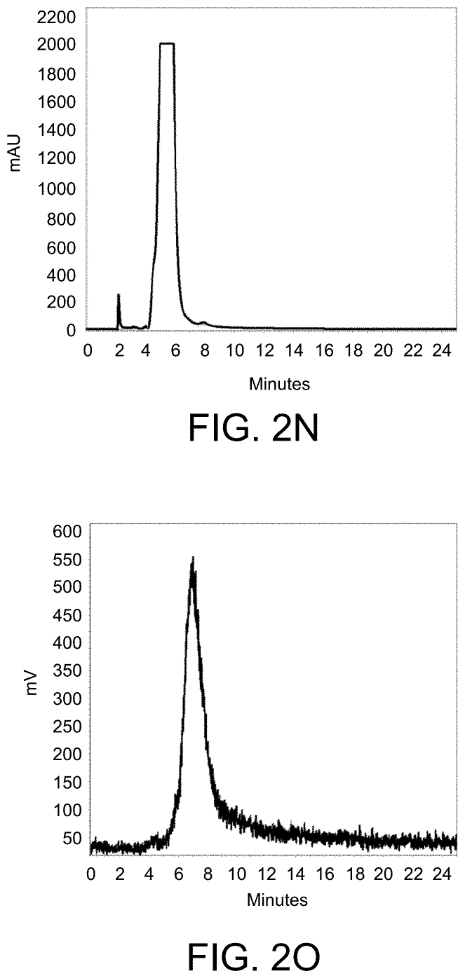

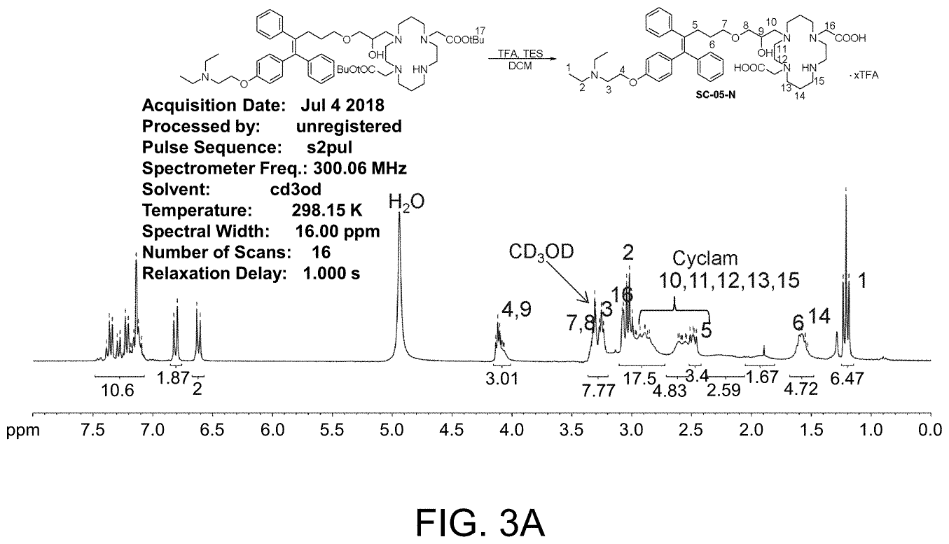

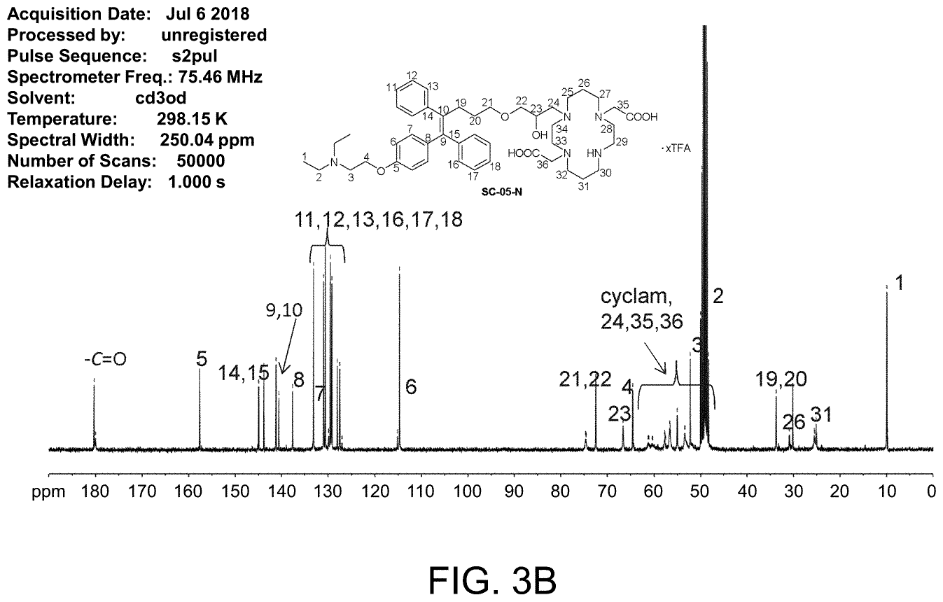

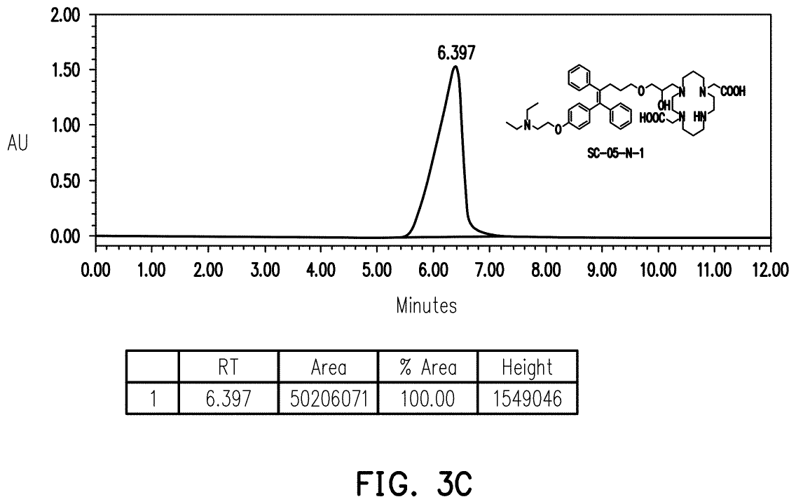

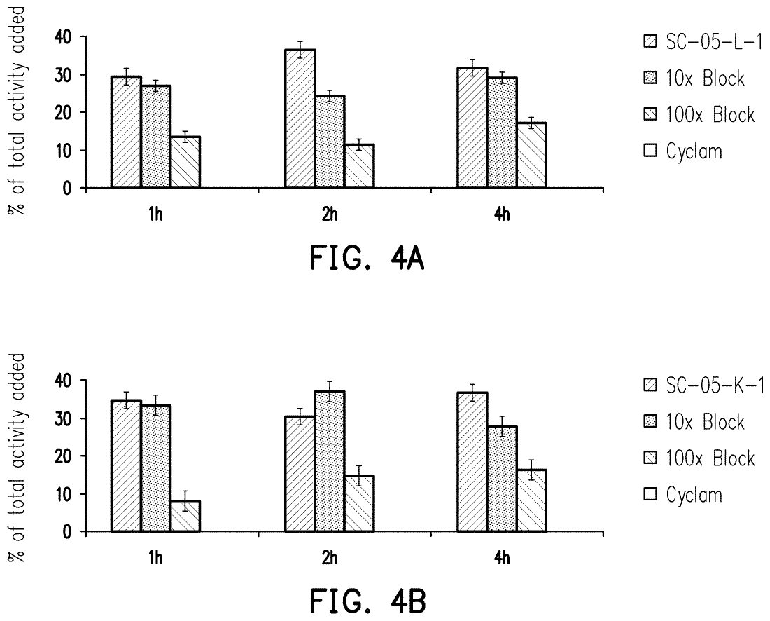

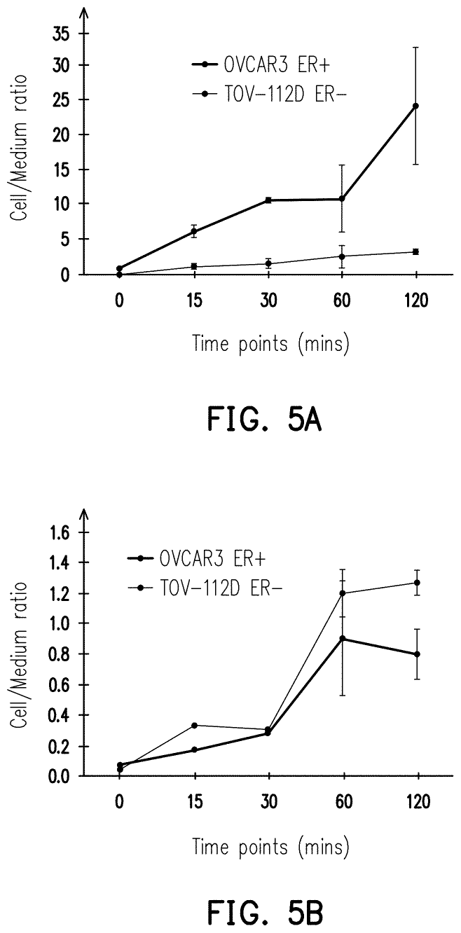

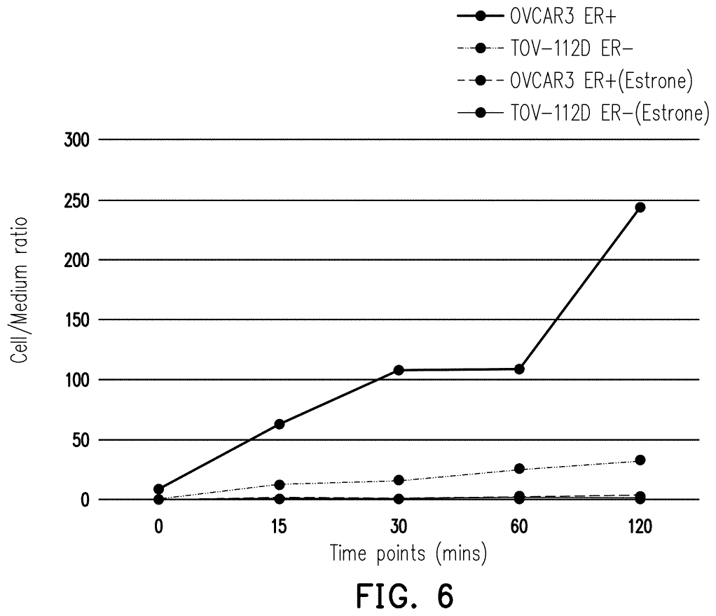

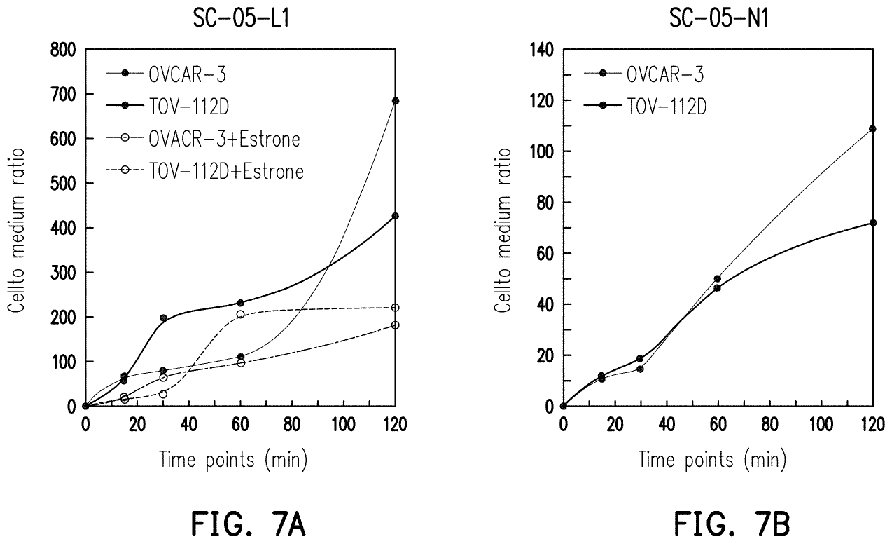

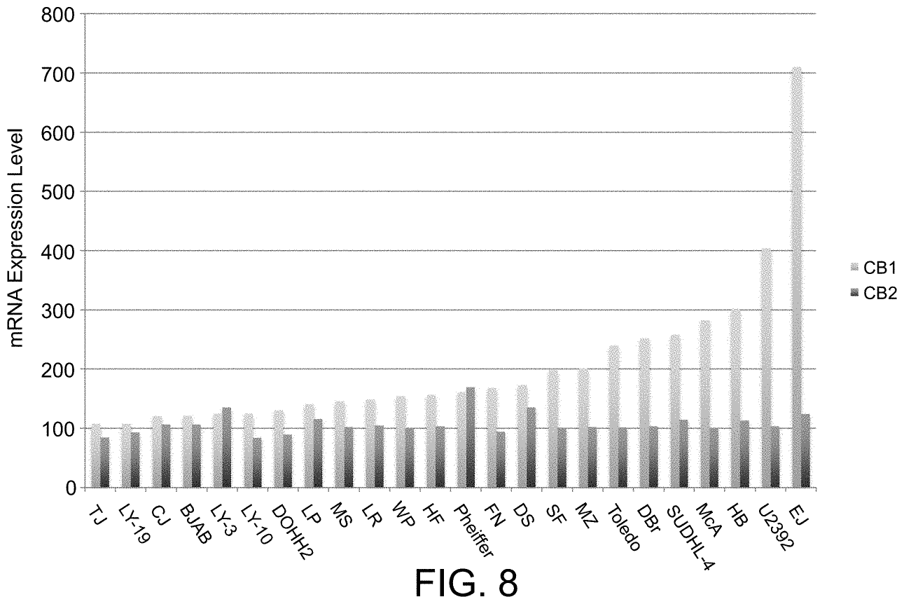

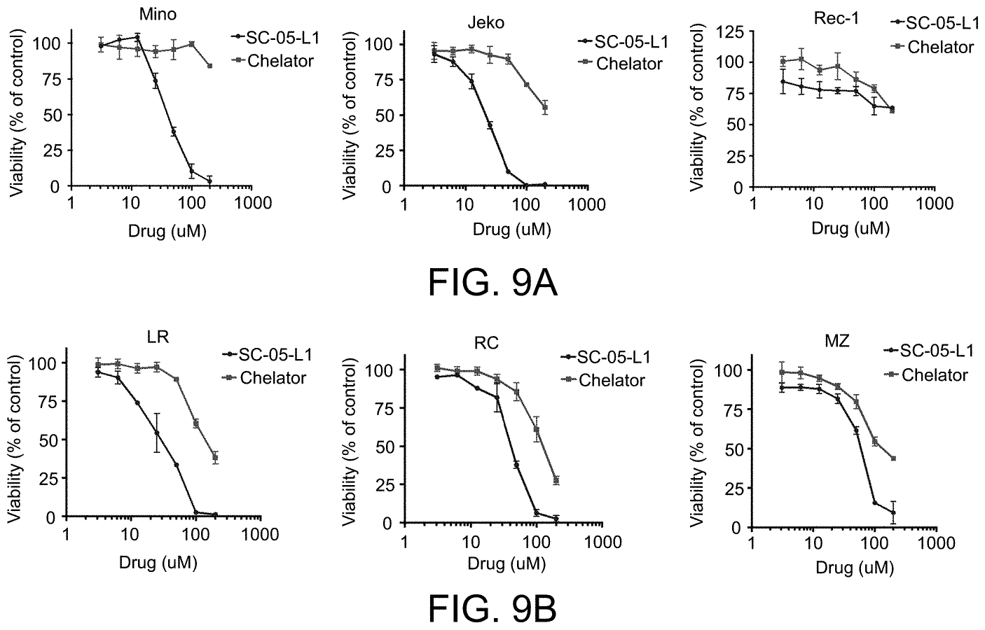

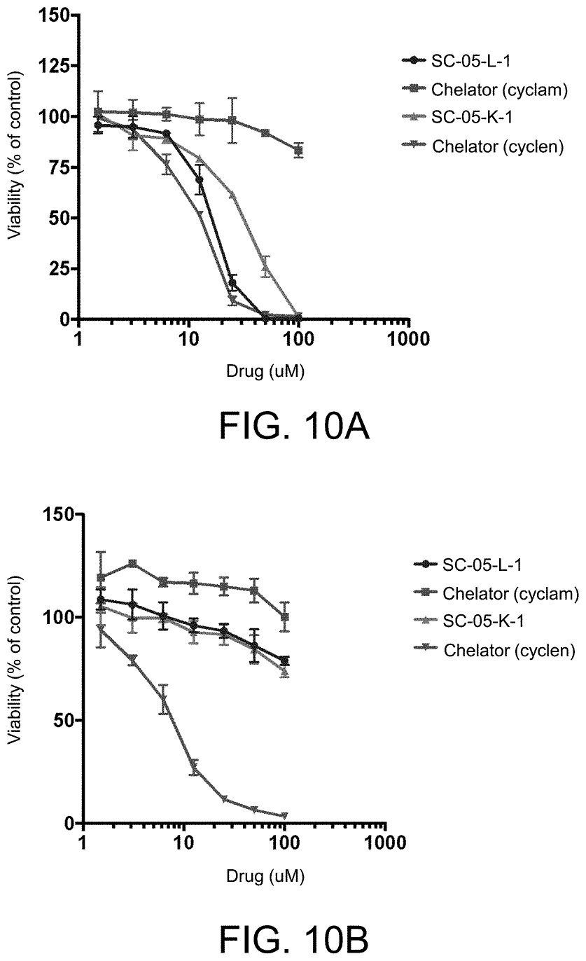

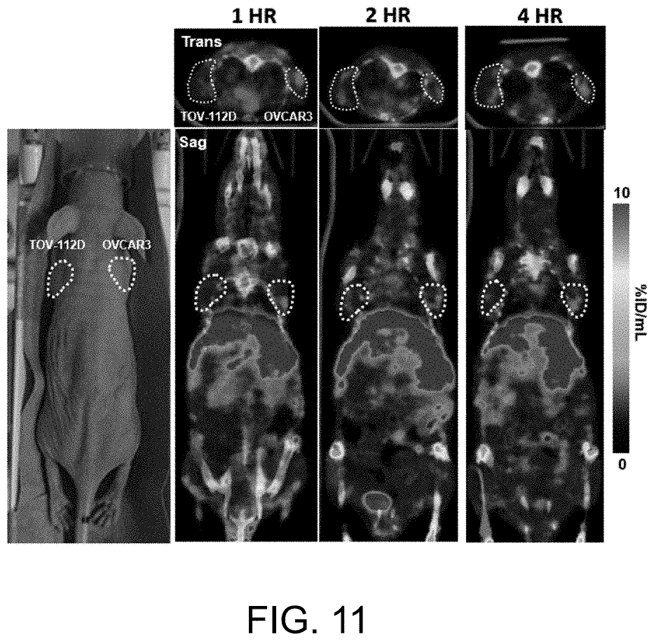

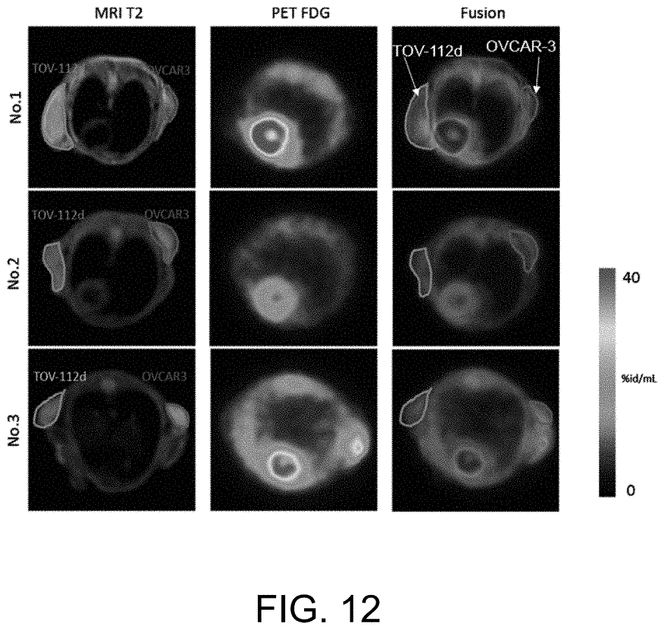

FIG. 2J and FIG. 2K show the radiochemical purity of Composition ^{99m}Tc-SC-05-L-1 synthesized in Example 4 of the invention in two different systems.

FIG. 2L and FIG. 2M show the labeling efficiency of Composition ^{99m}Tc-SC-05-L-1 synthesized in Example 4 of the invention in two different systems.

FIG. 2N and FIG. 2O show the in vitro stability of Composition ^{99m}Tc-SC-05-L-1 synthesized in Example 2 of the invention in two different systems.

FIG. 3A shows the ¹H-NMR spectrum of Compound SC-05-N-1 synthesized in Example 5 of the invention.

FIG. 3B shows the ¹³C-NMR spectrum of Compound SC-05-N-1 synthesized in Example 5 of the invention.

FIG. 3C shows the HPLC spectrum of Compound SC-05-N-1 synthesized in Example 5 of the invention.

FIG. 4A and FIG. 4B show the MCF-7 cell uptake and blocking studies of Composition ^{99m}Tc-SC-05-K-1 and Composition ^{99m}Tc-SC-05-L-1 synthesized in Example 2 and Example 4 of the invention.

FIG. 5A and FIG. 5B show the OVCAR3 cell and TOV-112D cell uptake studies of Composition ^{99m}Tc-SC-05-K-1 and Composition ^{99m}Tc-SC-05-L-1 synthesized in Example 2 and Example 4 of the invention.

FIG. 6 shows the OVCAR3 cell and TOV-112D cell uptake and blocking studies of Composition ^{99m}Tc-SC-05-L-1 synthesized in Example 2 of the invention.

FIG. 7A and FIG. 7B show the OVCAR3 cell and TOV-112D cell uptake studies of Composition ^{99m}Tc-SC-05-L-1 and Composition ^{99m}Tc-SC-05-N-1 synthesized in Example 4 and Example 5 of the invention.

FIG. 8 shows the effect of Composition SC-05-L-1 and Composition SC-05-K-1 against lymphoma cells of the invention.

FIG. 9A and FIG. 9B show the in vitro anti-cancer studies of Composition SC-05-L-1 synthesized in Example 3 of the invention.

FIG. 10A and FIG. 10B show the in vitro anti-cancer studies of Compound SC-05-K-1 and Compound SC-05-L-1 synthesized in Example 1 and Example 3 of the invention.

FIG. 11 shows the transaxial and whole-body planar imaging of mice bearing OVAC-3 and TOV-112D tumors administered with ^{99m}Tc-SC-05-N-1 synthesized in Example 5 of the invention.

FIG. 12 shows the transaxial images of mice bearing OVAC-3 and TOV-112D tumors in ¹⁸F-FDG group.

FIG. 13A and FIG. 13B shows the biodistribution of mice bearing OVAC-3 and TOV-112D tumors administered with ^{99m}Tc-SC-05-N-1 synthesized in Example 5 of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

Assessment of estrogen receptor-positive (ER+) pathway activated systems is the basis of hormone-dependent disease management. ER+ patients respond better to endocrine therapy and survived twice as long as negative ER patients. However, tumor resistance to antiestrogens is un-predictable. The drug resistance may be due to its poor or slow uptake by the tumor. A selective estrogen receptor modulator (SERM) could produce cross talk between ERs and cannabinoid receptors (CBR) pathway systems. Identify whether the SERM-based drug uses CBR as an active transport strategy could enhance drug to ER binding pocket and may overcome drug resistance. Accordingly, the present invention provides a composition for cross talk between the estrogen receptors and the cannabinoid receptors including a chelator and a receptor ligand.

In some embodiments, the chelator may be a nitrogen containing tetraazacyclic ring, for example. Specifically, the nitrogen containing tetraazacyclic ring may be a cyclam, a cyclen, a cyclam-carboxylic acid, or a cyclen-carboxylic acid, for example, but the invention is not limited thereto.

In some embodiments, the receptor ligand may be an estrogen ligand or an anti-estrogen ligand, for example. In some embodiments, the estrogen ligand may include estradiol, estrone, estiol, and clomiphene, for example. In some other embodiments, the anti-estrogen ligand may include, non-steroidal tamoxifen, torimiphene, raloxifen, and aminoglutethimide, for example. However, the invention is not limited thereto. In some embodiments, the receptor ligand has a spacer hydroxy group which will be described in detail below.

In some embodiments, the composition further includes a metal ion. Specifically, the metal ion may be a radionuclide, a non-radioactive metal, or a combination thereof, for example. In some embodiments, the radionuclide may be ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, ⁴⁵Ti, or a combination thereof, for example. In some other embodiments, the non-radioactive metal may be a technetium ion (Tc), a stannous ion (Sn), a copper ion (Cu), an indium ion (In), a thallium ion (Tl), a gallium ion (Ga), an arsenic ion (As), a rhenium ion (Re), a holmium ion (Ho), a yttrium ion (Y), a samarium ion (Sm), a selenium ion (Se), a strontium ion (Sr), a gadolinium ion (Gd), a bismuth ion (Bi), an iron ion (Fe), a manganese ion (Mn), a lutecium ion (Lu), a cobalt ion (Co), a platinum ion (Pt), a calcium ion (Ca), a rhodium ion (Rh), an europium ion (Eu), and a terbium ion (Tb), or a combination thereof, for example. However, the invention is not limited thereto. In one specific embodiment of the invention, the composition may be a ^{99m}Tc-cyclam-tamoxifen analogue. In another specific embodiment of the invention, the composition may be a ^{99m}Tc-cyclen-tamoxifen analogue.

It should be mentioned that the composition of the present invention may be used to identify the ER+ pathways through cell surface CBRs. Radiolabeled ER+ ligand is not only able to quantify ER+ tissue uptake to stage and re-stage of the cancer, but also able to select the patients for optimal response to therapy as well as to discontinue the treatment when resistance occurs. In other words, due to the structure of the composition, the composition may enhance drug to ER binding pocket by an active transport strategy, thereby overcoming the drug resistance.

The present invention further provides a method of synthesizing the composition. The steps of the synthesis method are described in detail below, but the invention is not limited thereto.

In some embodiments, the receptor ligand is conjugated to a tetracyclic ring with an epoxide first, for example. In other words, the epoxide is attached to an aliphatic chain of the receptor ligand. In some embodiments, the receptor ligand may be an estrogen agonist, an estrogen antagonist, or an aromatase inhibitor including non-steroidal derivatives of clomiphene, tamoxifen, raloxifene, torimiphene and aminoglutethimide, for example. In one specific embodiment of the invention, the anti-estrogen is tamoxifen, but the invention is not limited thereto. Specifically, a chlorinated epoxide (spacer) is reacted with aliphatic hydroxylated tamoxifen in an organic solvent, thereby producing the epoxide-tamoxifen. In this case, the receptor ligand is tamoxifen, a selective estrogen receptor modulator (SERM), could produce cross-talk between estrogen receptors and cannabinoid receptors pathway systems, but the invention is not limited thereto. Then, the epoxide-tamoxifen is reacted with a protected tetraazacyclic chelator including coupling agents. Thus, a hydroxy group is positioned at the chelator-tamoxifen conjugate in the finished product. In some embodiments, the tetraazacyclic chelators may be a cyclam or a cyclen, for example. However, the invention is not limited thereto. It should be noted that the hydroxyl group is located at the aliphatic chain of the receptor ligands. To be clearly understood, Scheme 1 shows a schematic diagram illustrating the receptor ligand (R) conjugated to the cyclam or the cyclen as shown below:

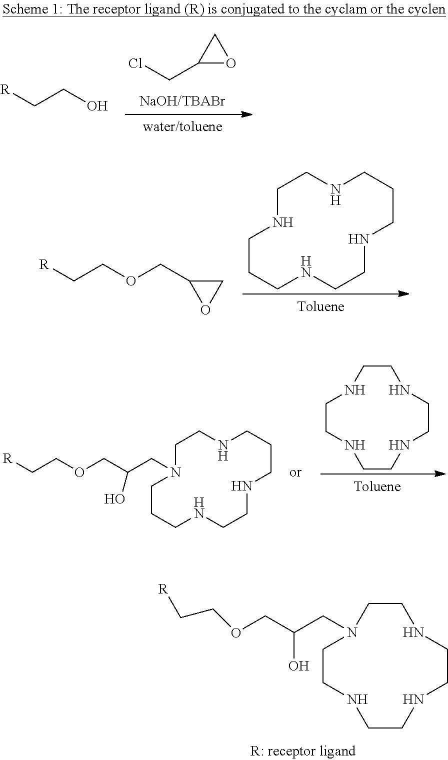

In some embodiments, a method of admixing may be carried out in an organic solvent, such as dimethylformamide, dimethylsulfoxide, dioxane, methanol, ethanol, hexane, methylene chloride, acetonitrile, tetrahydrofuran, or a mixture thereof. In other embodiments, the method of admixing may be carried out in an aqueous solvent. In some embodiments, one, two, or three of the nitrogen groups of the chelator may be protected, for example, by a tert-butyl or benzyl group, or unprotected.

In some embodiments, the method of the present invention may further include at least one purification step. Any compound of the present invention may be purified via any method known to those of skill in the art. Persons of skill in the art are familiar with such methods, and when those methods may be employed. For example, in a multi-step synthesis that is aimed at arriving at a particular compound, a purification step may be performed after every synthetic step, after every few steps, at various points during the synthesis, and/or at the very end of the synthesis. In some embodiments, one or more purification steps includes technique selected from the group consisting of silica gel column chromatography, HPLC (high-performance liquid chromatography) and LC (liquid chromatography). In certain embodiments, purification methods specifically exclude size exclusion chromatography and/or dialysis. It should be noted that the method of synthesizing the composition in organic solvents and the use of protecting groups, typically offer improvements in the purification of compounds. The installation of protecting groups permits various functional groups of intermediates during the synthesis to be protected, and facilitates the purification of those intermediates. Various means of purification using organic solvents allow for separation and isolation of desired compounds, such as imaging agents, with very little impurities. Thus, it is amenable to develop organic synthetic techniques to allow for site-specific conjugates of higher purities to be obtained in a more efficient way.

In one specific embodiment of the invention, the hydroxylated tamoxifen is conjugated to the cyclam and the cyclen at one nitrogen group using the synthetic route as shown in Scheme 2, Scheme 3 and Scheme 4 below. In this case, a hydroxy group is incorporated in the finished product. The protected chelator is used as to react an epoxylated tamoxifen to form a chelator-tamoxifen conjugate. The technology platform exploits conjugating antagonists and agonists and seeing their effects in various forms of diseases. In other words, the personalized technology platform may be designed on the basis of individual genetic make-up of cannabinoid receptors and estrogen receptors associated to each patient's disease. In other aspects, these synthesis methods may obviate the need of adding protecting groups to tamoxifen analogues and increase process efficiency and purify of the final product.

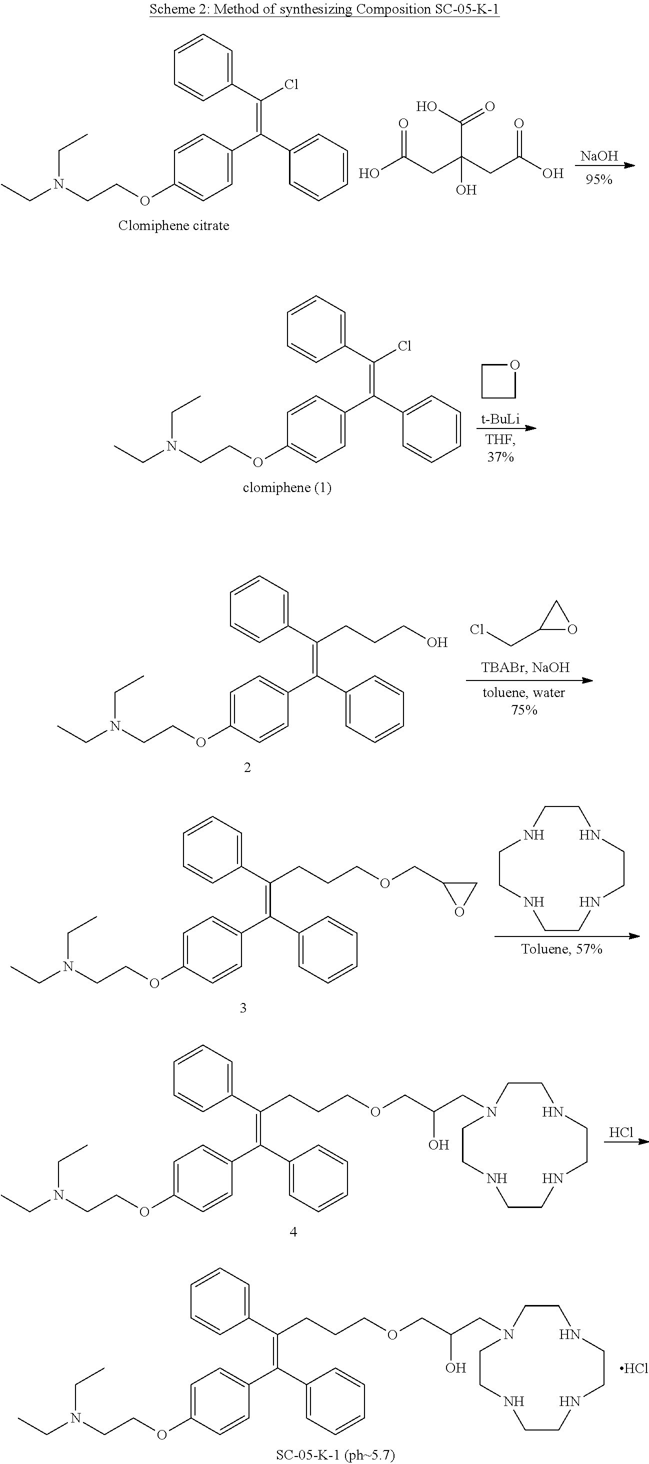

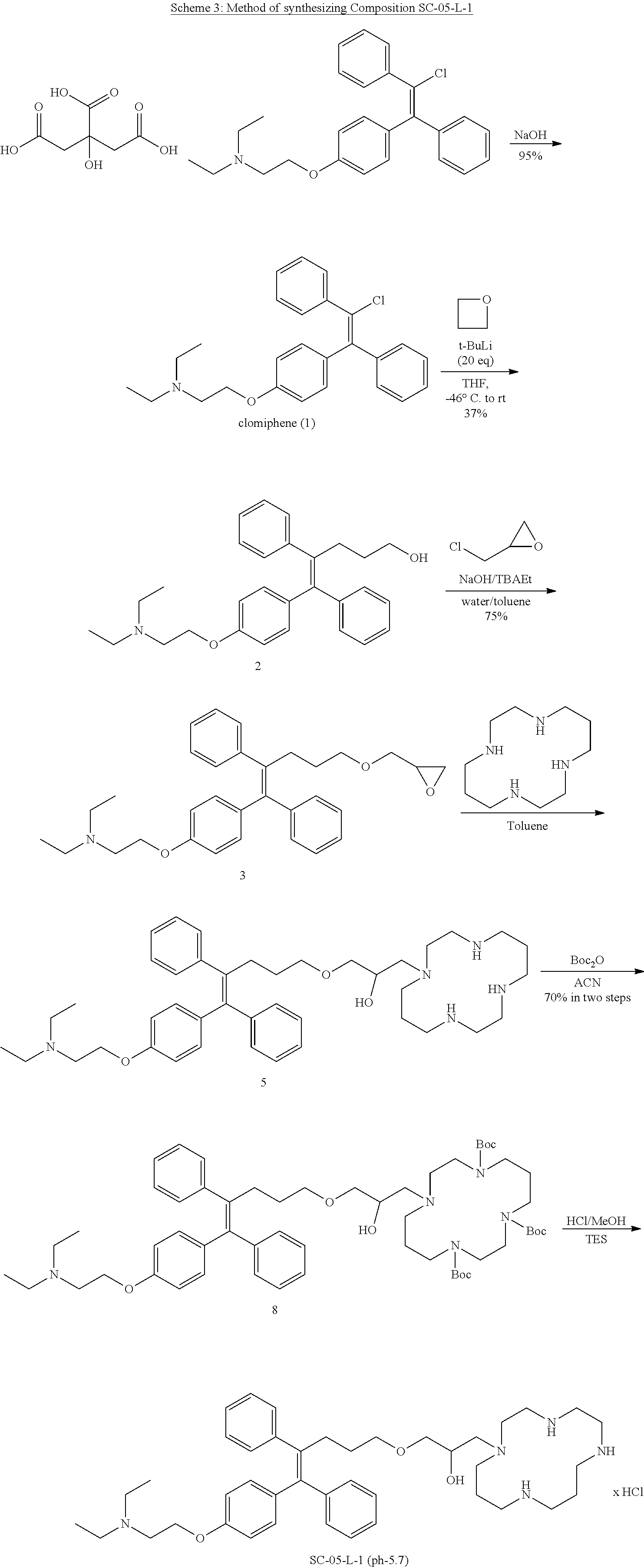

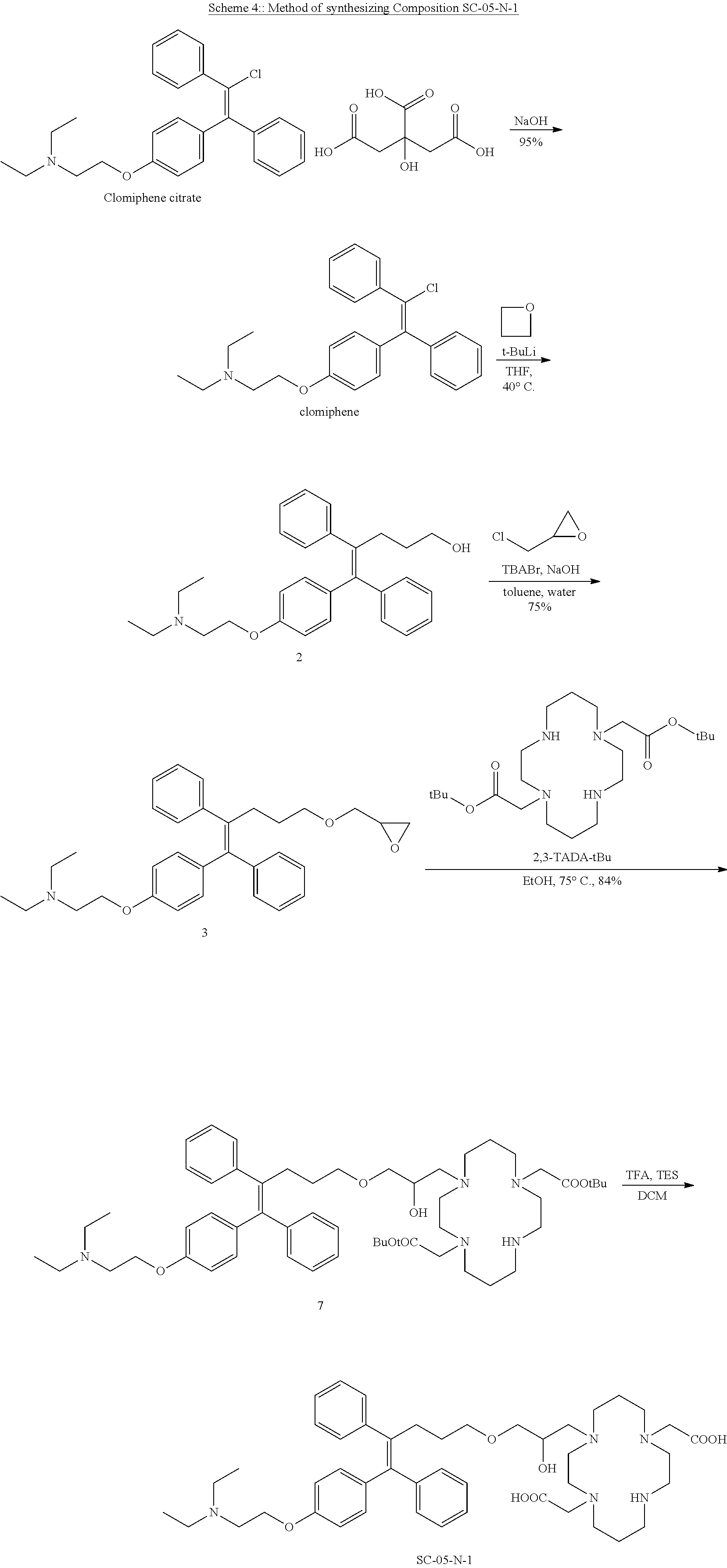

In addition, in some embodiments, pharmaceutical formulations or kits including the composition described above are provided. In other aspects, the composition may be further prepared in the pharmaceutical formulation or the kit using the chemical procedures known to skilled artisans. In some embodiments, the pharmaceutical formulation or the kit may further include antioxidants, stabilizing agents, preservatives or salts, for example. In some embodiments, the pharmaceutical formulation or the kit may include ascorbic acid, mannitol, tin (II) chloride and chelator-tamoxifen conjugate, for example. In some aspects, the pharmaceutical formulation or the kit may be an aqueous solution or a solution that has been frozen and/or lyophilized, for example. Herein, the “kit” is also called a “cold kit” in the field of molecular imaging.

Furthermore, the present invention accurately provides a method of imaging at the site of a disease in a given subject to perform a per/post treatment evaluation and to be able to monitor that subject for as long as that subject is being treated or under treatment with anti-estrogen. In certain aspects, the method includes detecting a signal generated by the radionuclide-labeled chelator-conjugates at the site of the disease of individual subjects, wherein a site of disease, if present, generates a signal that is more intense than surrounding the tissue. In some aspects, the metal ion may be a radionuclide and any radionuclide known to those of skill in art. In some embodiments, the radionucleodies include ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, and ⁴⁵Ti, for example, but the invention is not limited thereto. In other aspects, the metal ion may be a non-radioactive metal. In some embodiments, the site to be imaged may be a tumor or an ER-enriched tissue such as ovaries and uterine tissue. In some embodiments, the method may be defined as an imaging method for cancer, rheumatoid arthritis, osteoporosis, atherosclerosis, or endometrial tissue including administration of the composition described above. In one specific embodiment, the method may be defined as a method of imaging a site within a subject including detecting a signal from metal ion labeled chelator-receptor ligand conjugate that is localized at the site, but the invention is not limited thereto. In some embodiments, the signal may be detected using a technique selected from the group consisting of PET, PET/CT, SPECT, SPECT/CT, PET/MRI, SPECT/MRI, and an optical imaging hybrid with nuclear imaging device, for example. In other embodiments, the image may be a gamma image, a PET image, a PET/CT image, a SPECT image, a SPECT/CT image, a PET/MRI image, a SPECT/MRI image, or a hybrid image, for example. It should be noted that the composition described above may be made as a kit for imaging, and an imaging dose is defined as the kit. Besides, the method may be further defined as a method of treating a subject with cancer or endometriosis. In particular aspects, the cancer is breast cancer, lung cancer, prostate cancer, ovarian cancer, uterine cancer, cervical cancer, or endometrial cancer, for example, but the invention is not limited thereto. In some embodiments, the method may be defined as a treatment method for cancer, rheumatoid arthritis, osteoporosis, atherosclerosis, or endometrial tissue including administration of the composition described above, for example. In other words, there is provided a method of imaging a site, diagnosing a disease, or treating a disease within a subject including administering a metal ion labeled-chelator-receptor ligand conjugate to the subject, wherein the site is imaged, the disease is diagnosed, or the disease is treated.

On the other hand, it should be noted that the composition of the invention may be applied to molecular imaging and therapy. For example, the composition of the invention may be used as a molecular nuclear imaging agent. Specifically, the molecular nuclear imaging agent enables the comprehensive characterization of therapeutic intervention and can be used in patient selection, pharmacokinetic, dosage-finding and proof-of-concept studies. The effort in receptor image-guided cell therapy approaches in parallel with instrumentation development would be more comprehensive in the outcome assessment of patient response to treatment. More specifically, the molecular imaging agent using chelation provides advantages in batch-to-batch reproducibility of radiochemical yield, purity, production cost and the availability of the agent in routine clinical practice.

In addition, the invention technology platform integrates a metal ion, chelator, and receptor ligand. The receptor ligand may be used as a homing agent, which plays a dual role by cross talking between cell surface receptors and intracellular cytosolic receptors, thus, enhance cell uptake of the homing agent. For instance, CB1/CB2 receptor and ER pathways are overlapped in various cancers. Tamoxifen is known to provide cross-talk between ER and CBRs. Thus, it would be ideal to develop a tamoxifen-based imaging agent to measure ER systems activity via CB1/CB2 receptors. Such a tamoxifen-based imaging would help to monitor CB1/CB2 receptor and ER pathway-directed treatment response as well as predict the selection of patients for optimal treatment response. In this case, a hydroxy group was incorporated at the aliphatic spacers in chelator-tamoxifen conjugates to allow phosphorylation during diagnostic imaging with innovative tools to understand the dynamic changes in pathway-activated cell receptors leading to tissue degeneration, inflammatory, and proliferative disorders and to improve patient diagnosis, therapy and prognosis. However, the invention is not limited thereto.

To prove that the compositions of the present invention are suitable for imaging and be used for cancer therapy, the compositions of the present invention are synthesized and tested by using the method described in the following examples.

### Example 1

### Synthesis of Compound SC-05-K-1

In this example, 4 specific compounds (Compounds 1 to 4) and Compound SC-05-K-1 of the present invention were synthesized.

A. Synthesis of Compound 1

2N NaOH solution (10 mL) was added to a solution of clomiphene citrate (1 g, 1.69 mmol) and ethyl acetate (EA, 10 mL) at room temperature. The mixture was stirred vigorously for 30 min and extracted with EA three times (10 mL, 8 mL, 6 mL). The organic layer was concentrated under reduce pressure to give free-base clomiphene (Compound 1, 685.7 mg, 1.68 mmol, 99%) as a colorless oil.

B. Synthesis of Compound 2

tert-Butyl lithium (50 mL, 96 mmol, 1.9 M in pentane) was added drop wisely to a solution of Compound 1 (1.95 g, 4.8 mmol) in tetrahydrofuran (THF, 50 mL) at −40° C. Trimethylene oxide (6.26 mL, 96 mmol) was added drop wisely and the mixture was stirred at −40° C. for 30 min. The reaction was warmed to room temperature and stirred continuously at room temperature for 18 hr. Water was added to reaction carefully and the reaction was extracted with EA three times (50 mL, 30 mL, 20 mL). The EA layer was dried over anhydrous magnesium sulfate. After filtration, the EA solvent was concentrated under reduced pressure. The crude product was purified by column chromatography (EA/hexane/TEA, 1/3/0.1) to give (Z)-5-(4-(2-(diethylamino)ethoxy)phenyl)-4,5-diphenylpent-4-en-1-ol (Compound 2, 671.5 mg, 1.6 mmol, 36%) as a white solid.

C. Synthesis of Compound 3

To a suspension of Compound 2 (503.3 mg, 1.17 mmol) in 35% NaOH solution (12 mL), tetrabutyl ammonium bromide (TBABr, 113.3 mg, 0.35 mmol) was added. The reaction mixture was stirred vigorously. Epichlorohydrin (758.7 mg, 8.2 mmol) and few drops of toluene were then added to reaction. The reaction mixture was stirred at room temperature for 15 hr. EA (20 mL) was added to reaction and the reaction was extracted three times (15 mL, 10 mL). The organic layer was dried over magnesium sulfate. After filtration, the solvent was concentrated under reduced pressure and the crude product was purified by column chromatography (EA/hexane/TEA, 1/3/0.1) to give (Z)-N,N-diethyl-2-(4-(5-(oxiran-2-ylmethoxy)-1,2-diphenylpent-1-en-1-yl)phenoxy)ethan-1-amine (Compound 3, 432.1 mg, 0.89 mmol, 76%) as a yellow oil.

D. Synthesis of Compound 4

A mixture of 1,4,7,10-tetraazacyclododecane (cyclen, 642.9 mg, 3.73 mmol) and Compound 3 in toluene (4 mL) was heated to 100° C. until all the cyclen dissolved. The reaction mixture was stirred at 100° C. for 16 hr. The reaction was cooled to room temperature and kept in refrigerator for 3 hr. The precipitate of excess cyclen was then removed by filtration and washed with cold toluene. The toluene filtrates were combined and concentrated. The crude product was purified by column chromatography (DCM/MeOH/NH₄OH, 1/1/0.1) to give (Z)-1-(1,4,7,10-tetraazacyclododecan-1-yl)-3-((5-(4-(2-(diethylamino)ethoxy)phenyl)-4,5-diphenylpent-4-en-1-yl)oxy)propan-2-ol (Compound 4, 330 mg, 0.50 mmol, 67%) as a yellow oil.

E. Synthesis of Compound SC-05-K-1

1N HCl solution was added to a mixture of 4 (330 mg, 0.50 mmol) and water (1 mL) was added drop wisely until the pH value is 5-7. The mixture was then purified by reverse phase column chromatography to give pure Compound SC-05-K-1 (175 mg) as a white solid.

### Characterization of Compounds 1-3 and Compound SC-05-K-1

NMR data was collected from 500 MHz Varian Inova NMR spectrometer (Palo Alto, Calif.) equipped with 5 mm PFG Triple ¹H-¹³C-¹⁵N probe, 5 mm PFG ¹H-¹⁹C-¹⁵N-³¹P switchable probe and 4 mm ¹H-¹³C Nano probe. Mass Spectrometry was obtained from Bruker Solarix (Germany). HPLC data was collected from Waters 2695 Separations Module (Milford, Mass.) equipped with PC HILIC Column, (5 μm, 2.0 mm I.D.×150 mm).

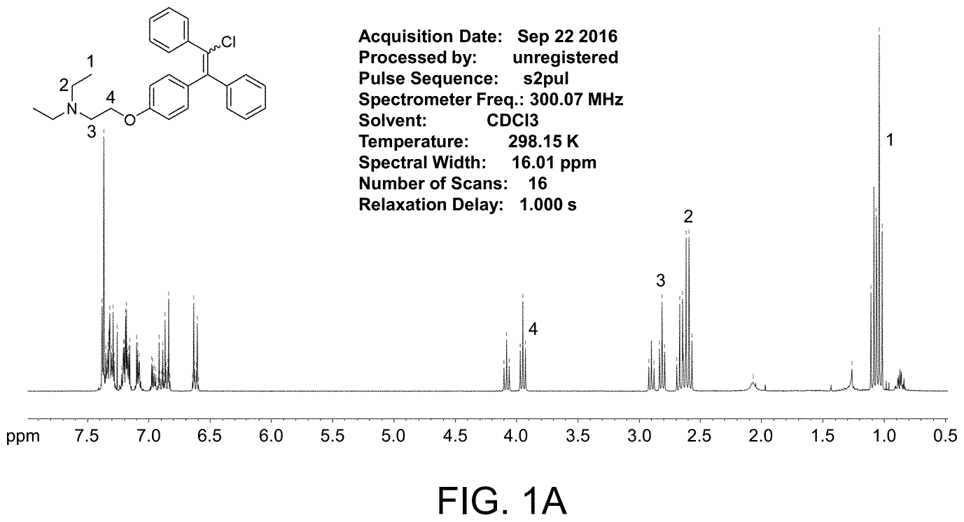

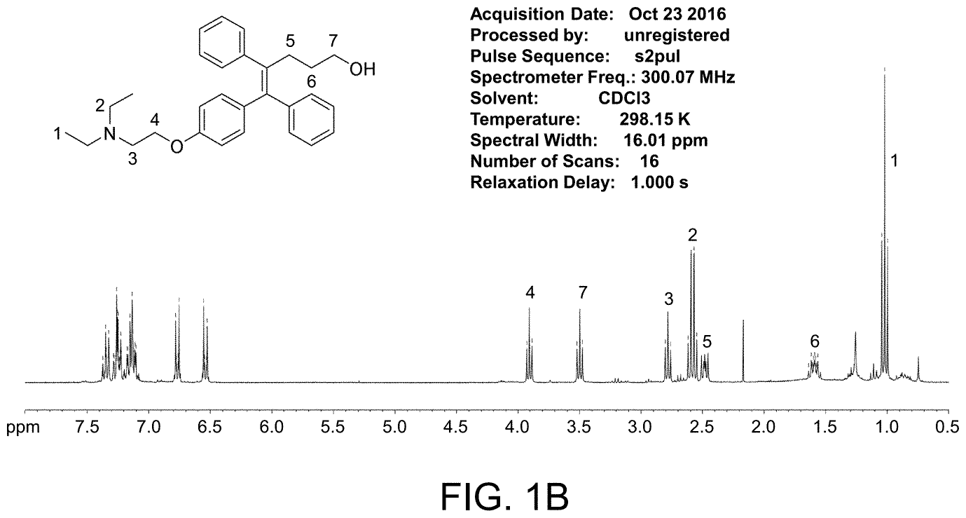

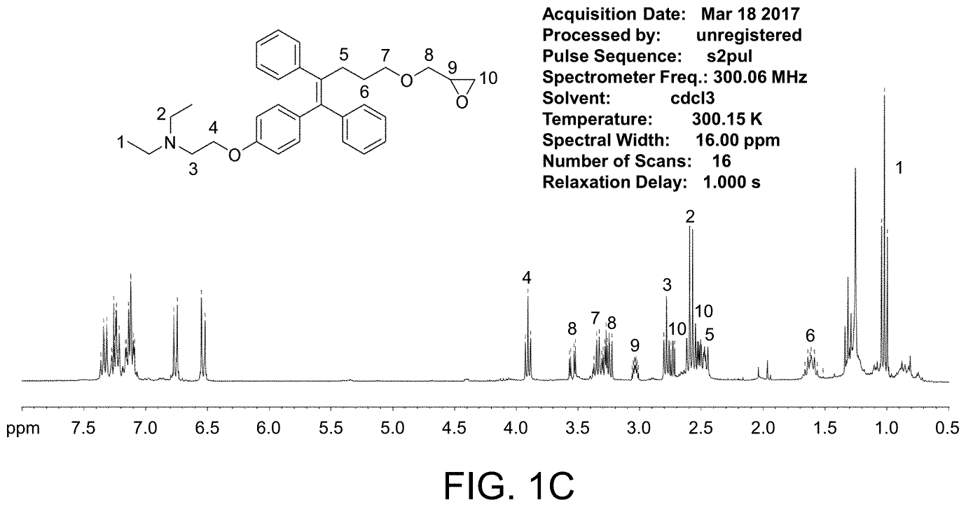

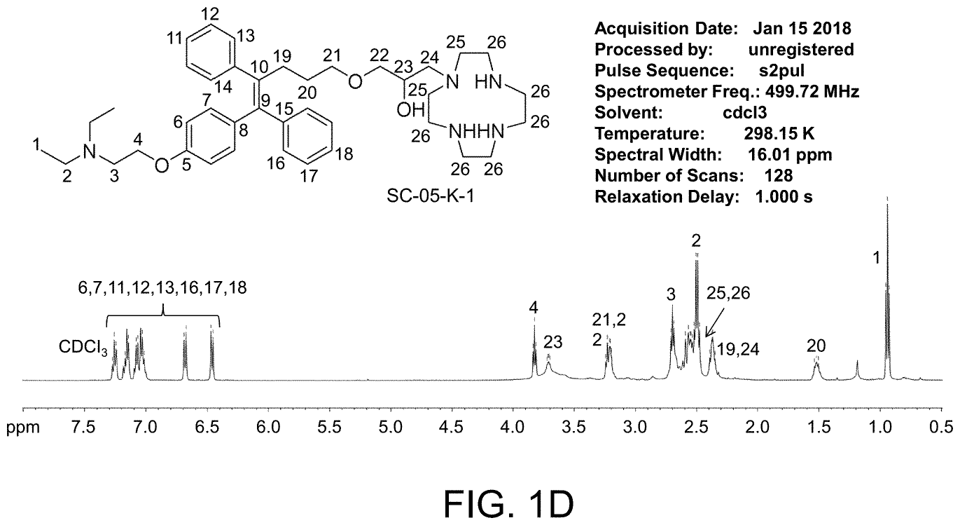

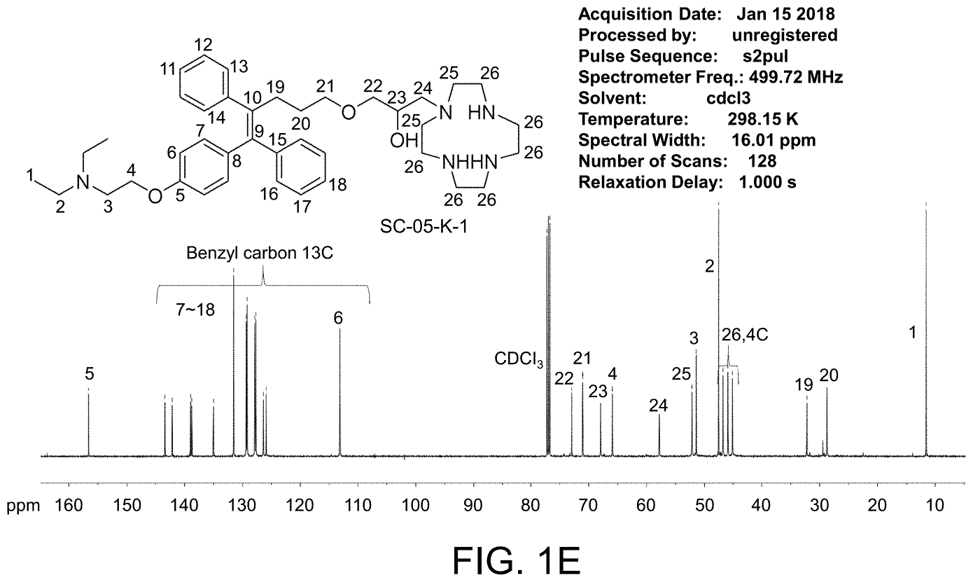

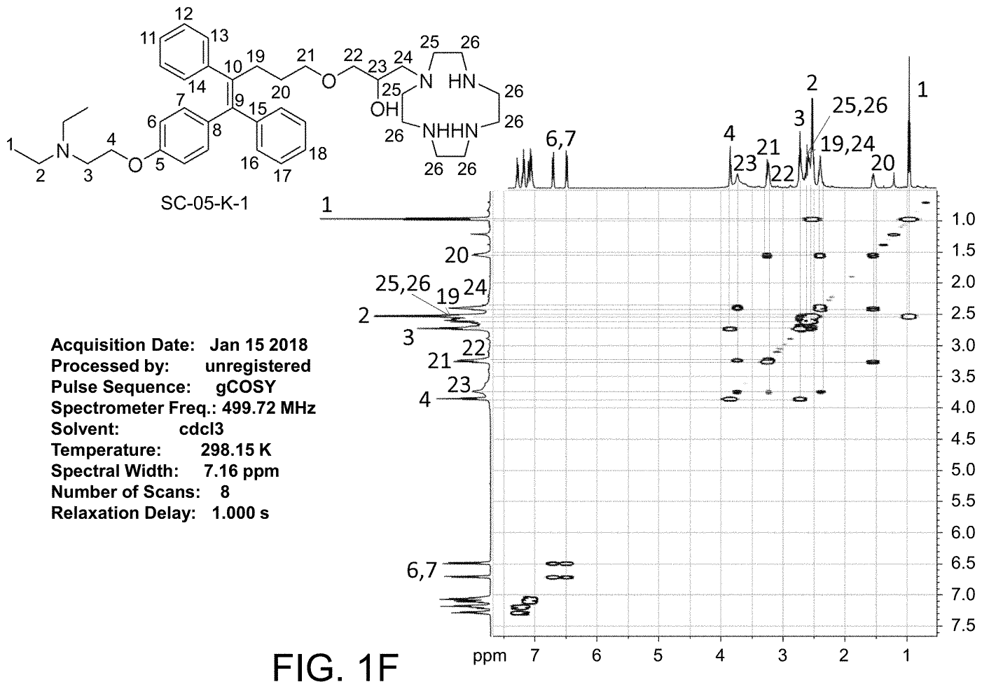

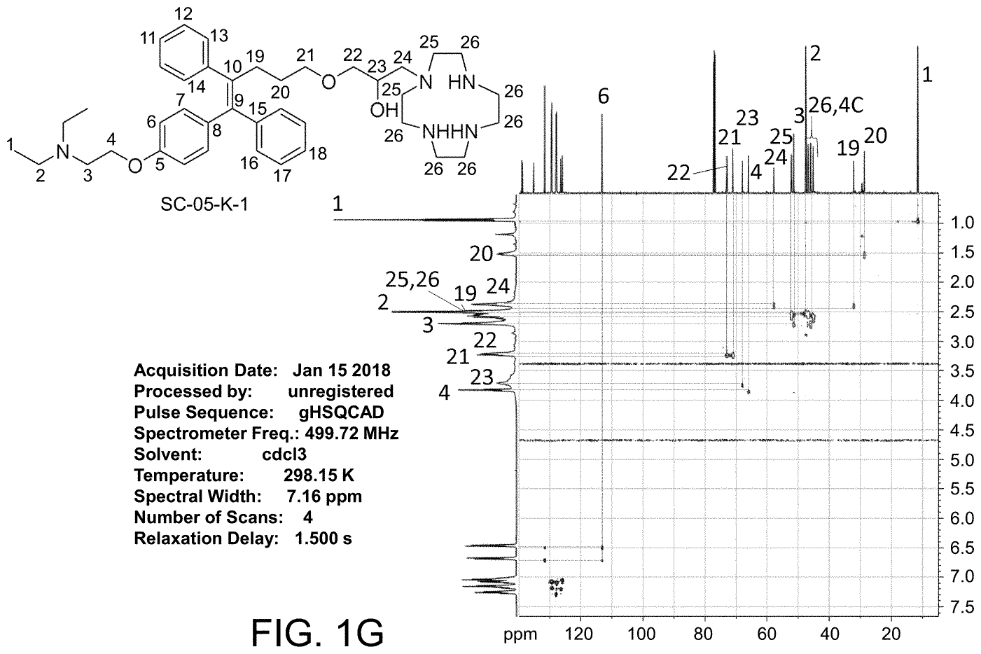

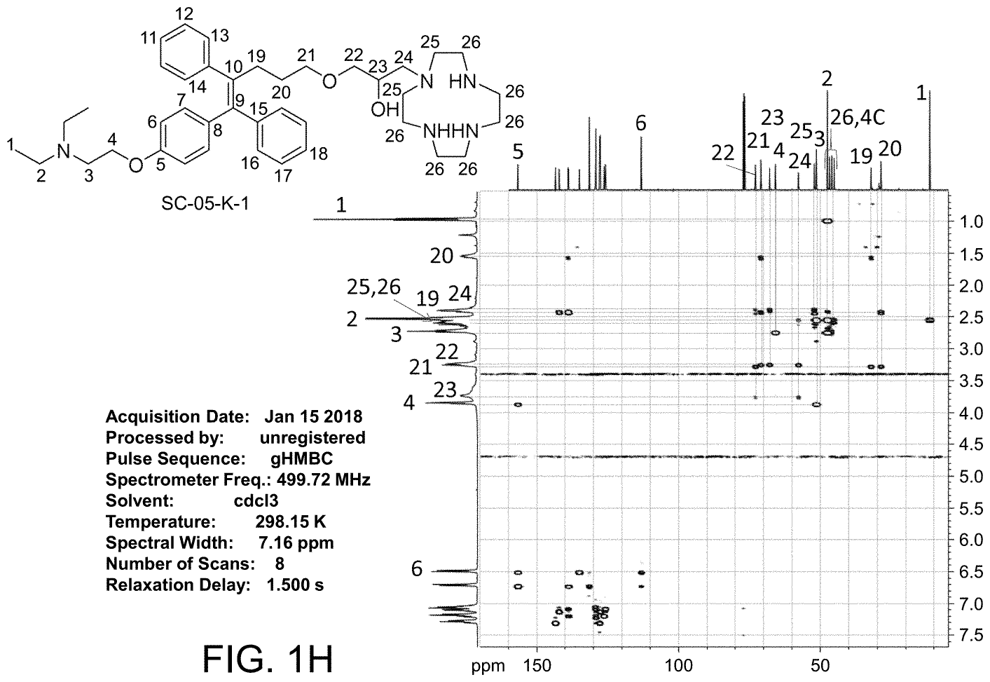

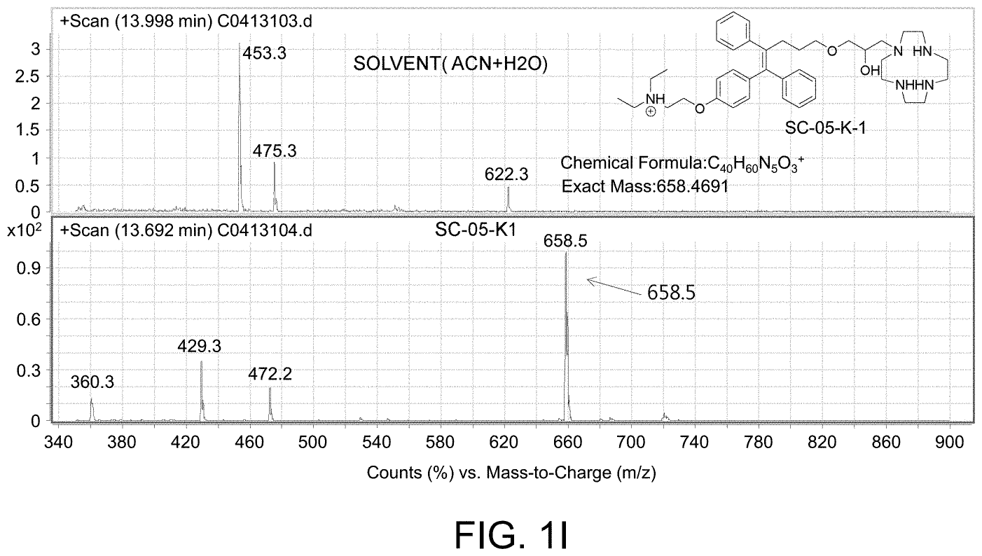

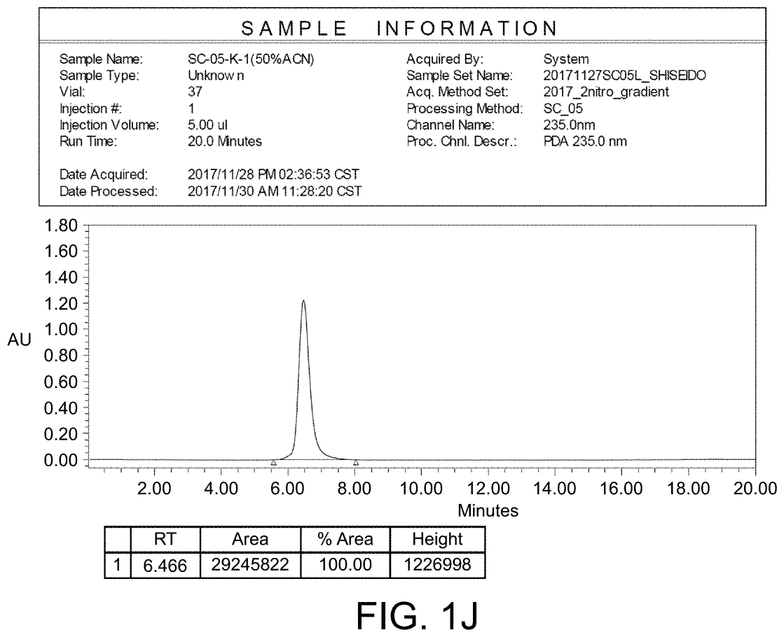

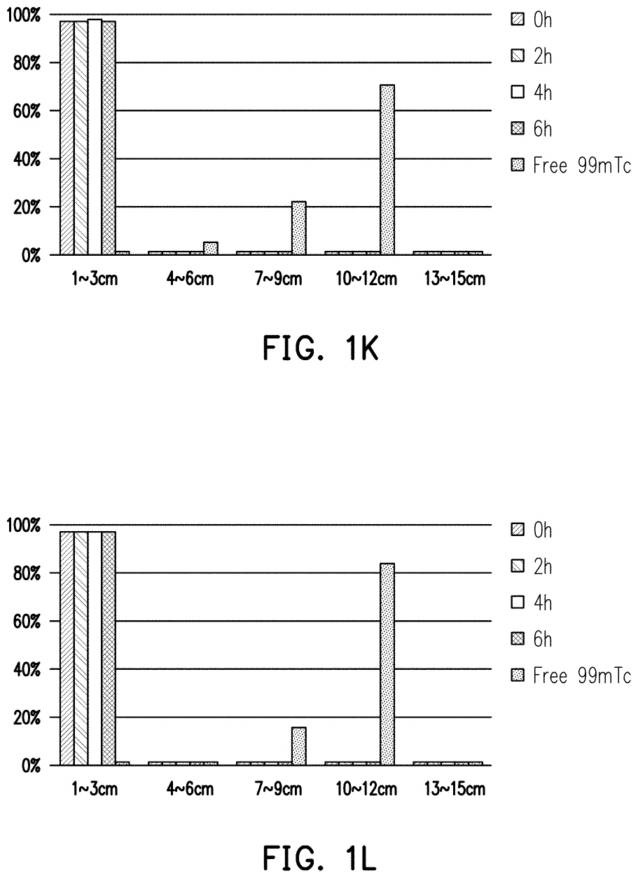

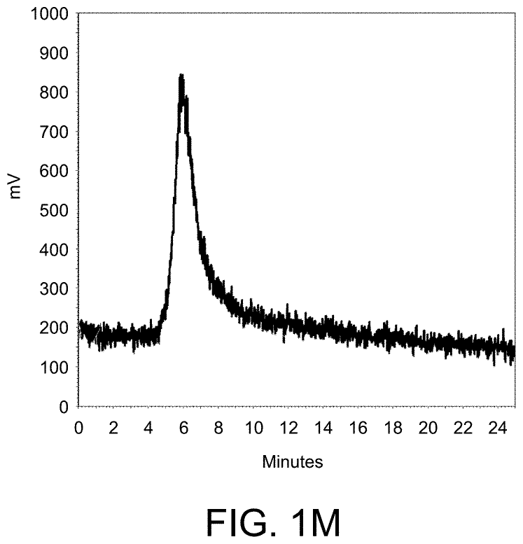

FIG. 1A shows the ¹H-NMR spectrum of Compound 1 synthesized in Example 1 of the invention. FIG. 1B shows the ¹H-NMR spectrum of Compound 2 synthesized in Example 1 of the invention. FIG. 1C shows the ¹H-NMR spectrum of Compound 3 synthesized in Example 1 of the invention. The structures of Compounds 1-3 were confirmed by ¹H-NMR, and the analysis results are presented in FIG. 1A to FIG. 1C respectively.

FIG. 1D shows the ¹H-NMR spectrum of Compound SC-05-K-1 synthesized in Example 1 of the invention. FIG. 1E shows the ¹³C-NMR spectrum of Compound SC-05-K-1 synthesized in Example 1 of the invention. FIG. 1F shows the ¹H-,¹H COSY NMR spectrum of Compound SC-05-K-1 synthesized in Example 1 of the invention. FIG. 1G shows the ¹H-,¹³C HSQC NMR spectrum of Compound SC-05-K-1 synthesized in Example 1 of the invention. FIG. 1H shows the ¹H-,¹³C HMBC NMR spectrum of Compound SC-05-K-1 synthesized in Example 1 of the invention. FIG. 1I shows the LC-MS spectrum of Compound SC-05-K-1 synthesized in Example 1 of the invention. FIG. 1J shows the HPLC spectrum of Compound SC-05-K-1 synthesized in Example 1 of the invention. The structure of Compound SC-05-K-1 was confirmed by ¹H-NMR, ¹³C-NMR, ¹H-,¹H COSY NMR, ¹H-,¹³C HSQC NMR and ¹H-,¹³C HMBC NMR, and the analysis results are presented in FIG. 1D to FIG. 1H respectively. Also, Compound SC-05-K-1 was analyzed using mass spectrometry and the results are presented in FIG. 1I and FIG. 1J. As shown in FIG. 1J, HPLC analysis of Compound SC-05-K-1 (pH 5-7) using HILIC column shows the retention time around 6.5 min.

### Example 2

Synthesis of Composition ^{99m}Tc-SC-05-K-1

Sodium pertechnetate (Na^{99m}TcO₄) was obtained from ⁹⁹Mo/^{99m}Tc generator by Covidien (Houston, Tex.). Radiosynthesis of Composition ^{99m}Tc-SC-05-K-1 was achieved by adding ^{99m}Tc-pertechnetate (40-50 mCi) into the lyophilized residue of Compound SC-05-K-1 (5 mg) and tin (II) chloride (SnCl₂, 100 μg). The complexation of Compound SC-05-K-1 with ^{99m}Tc was carried out at pH 6.5.

Characterization of Composition ^{99m}Tc-SC-05-K-1

Radiochemical purity was determined by TLC (Waterman No.1, Aldrich-Sigma, St. Louis, Mo.) eluted with acetone and saline. High-performance liquid chromatography (HPLC), equipped with a NaI detector and UV detector (235 nm), was performed on a PC HILIC Column (2.0 mm I.D.×150 mm, Agilent, Santa Clara, Calif.) eluted with acetonitrile/water (1:1 V/V) at a flow rate of 0.5 mL/min.

FIG. 1K and FIG. 1L show the radiochemical purity of Composition ^{99m}Tc-SC-05-K-1 synthesized in Example 2 of the invention in two different systems. Specifically, FIG. 1K shows the radiochemical purity of Composition ^{99m}Tc-SC-05-K-1 in an acetone system, and FIG. 1L shows the radiochemical purity of Composition ^{99m}Tc-SC-05-K-1 in a saline system. As shown in FIG. 1K and FIG. 1L, the radiochemical purity of Composition ^{99m}Tc-SC-05-K-1 (stayed at origin) was greater than 95% with Rf value 0.1 up to 6 hr, wherein free Na^{99m}TcO₄ was migrated to solvent front.

FIG. 1M shows the labeling efficiency of Composition ^{99m}Tc-SC-05-K-1 synthesized in Example 2 of the invention. Specifically, Composition SC-05-K-1 (5 mg in 100 μL saline) was added 100 μg tin (II) chloride (in 100 μL H₂O) followed by 200 μL Na^{99m}TcO₄⁻ (˜5 mCi). As shown in FIG. 1M, HPLC analysis of Composition ^{99m}Tc-SC-05-K-1 shows the retention time around 6.5 min.

### Example 3

### Synthesis of Compound SC-05-L-1

In this example, 5 specific compounds (Compounds 1-3, 5 and 6) and Compound SC-05-L-1 of the present invention were synthesized. The synthesis of Compounds 1-3 are similar to that of Compounds 1-3 described above, and are not repeated herein.

F. Synthesis of Compound 5

To a round bottom flask, Compound 3 (500 mg, 1.0295 mmol), 1,4,8,11-tetraazacyclotetradecane (cyclam, 1040 mg, 5.140 mmol) were dissolved toluene (5 mL). Reaction solution was heated to 100° C. and refluxed overnight. The reaction mixture was then cooled to −20° C. The precipitate was removed by filtration and the filtrate was collected, dried over magnesium sulfate, filtered and the solvent was concentrated under vacuum to afford crude product (Z)-1-(1,4,8,11-tetraazacyclotetrade can-1-yl)-3-((5-(4-(2-(diethylamino)ethoxy)phenyl)-4,5-diph enylpent-4-en-1-yl)oxy)propan-2-ol (Compound 5). Compound 5 was directly used in next step without further purification.

G. Synthesis of Compound 6

To a suspension of Compound 5 (600 mg, 0.8746 mmol) in acetonitrile (10 mL), di-tert-butyl dicarbonate (1.53 g, 7.0103 mmol) was added drop wisely at room temperature.

Reaction suspension was stirred overnight and gradually turn homogenous. As the reaction was completed, solution was concentrated under vacuum then purified by column chromatography with eluent hexane/ethylacetate/triethylamine=4/1/0.1 to give tri-tert-butyl (Z)-11-(3-((5-(4-(2-(diethylamino)ethoxy)phenyl)-4,5-diphenylpent-4-en-1-yl)oxy)-2-hydroxypropyl)-1,4,8,11-tetraazacyclotetradecane-1,4,8-tricarboxylate (Compound 6) as yellow sticky oil (two steps yield 70%).

H. Synthesis of Compound SC-05-L-1

To a round bottom flask of compound 5 (800 mg, 0.8111 mmol), triethylsilane (1.3 mL, 8.139 mmol) was added, followed by HCL (10 mL) in methanol (10 mL). Reaction solution was stirred at room temperature for 4 hr and monitored by TLC. As the reaction completed, solution was concentrated under vacuum, purified by reverse phase column chromatography with eluent from water to methanol to afford light yellow solid product (Z)-1-(1,4,8,11-tetraazacyclotetrade can-1-yl)-3-((5-(4-(2-(diethylamino)ethoxy)phenyl)-4,5-diphenylpent-4-en-1-yl)oxy)propan-2-ol hydrochloride salt (Compound SC-05-L-1, 637 mg).

### Characterization of Compounds 5 and 6 and Compound SC-05-L-1

NMR data was collected from 500 MHz Varian Inova NMR spectrometer (Palo Alto, Calif.) equipped with 5 mm PFG Triple ¹H-¹³C-¹⁵N probe, 5 mm PFG ¹H-¹⁹C-¹⁵N-³¹P switchable probe and 4 mm ¹H-¹³C Nano probe. Mass Spectrometry was obtained from Bruker Solarix (Germany). HPLC data was collected from Waters 2695 Separations Module (Milford, Mass.) equipped with PC HILIC Column, (5 μm, 2.0 mm I.D.×150 mm).

FIG. 2A shows the ¹H-NMR spectrum of Compound 5 synthesized in Example 3 of the invention. FIG. 2B shows the ¹H-NMR spectrum of Compound 6 synthesized in Example 3 of the invention. The structure of Compounds 5 and 6 were confirmed by ¹H-NMR, and the analysis results are presented in FIG. 2A and FIG. 2B respectively.

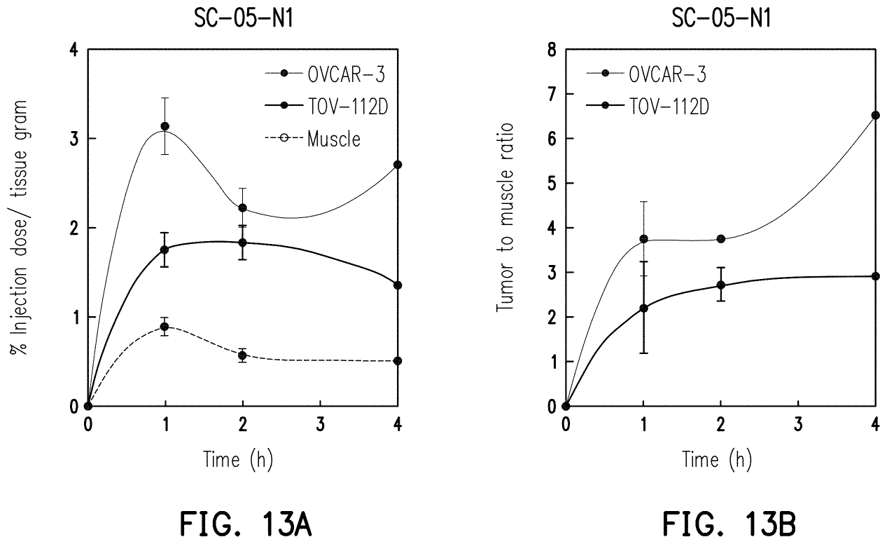

FIG. 2C shows the ¹H-NMR spectrum of Compound SC-05-L-1 synthesized in Example 3 of the invention. FIG. 2D shows the ¹³C-NMR spectrum of Compound SC-05-L-1 synthesized in Example 3 of the invention. FIG. 2E shows the ¹H-,¹H COSY NMR spectrum of Compound SC-05-L-1 synthesized in Example 3 of the invention. FIG. 2F shows the ¹H-, ¹³C HSQC NMR spectrum of Compound SC-05-L-1 synthesized in Example 3 of the invention. FIG. 2G shows the ¹H-,¹³C HMBC NMR spectrum of Compound SC-05-L-1 synthesized in Example 3 of the invention. FIG. 2H shows the LC-MS spectrum of Compound SC-05-L-1 synthesized in Example 3 of the invention. FIG. 21 shows the HPLC spectrum of Compound SC-05-L-1 synthesized in Example 3 of the invention. The structure of Compound SC-05-L-1 was confirmed by ¹H-NMR, ¹³C-NMR, ¹H-,¹H COSY NMR, ¹H-,¹³C HSQC NMR and ¹H-,¹³C HMBC NMR, and the analysis results are presented in FIG. 2C to FIG. 2G. Also, Compound SC-05-L-1 was analyzed using mass spectrometry and the results are presented in FIG. 2H and FIG. 21. As shown in FIG. 21, HPLC analysis of Compound SC-05-L-1 shows the retention time around 6.3 min.

### Example 4

Synthesis of Composition ^{99m}Tc-SC-05-L-1

Sodium pertechnetate (Na^{99m}TcO₄) was obtained from ⁹⁹Mo/^{99m}Tc generator by Covidien (Houston, Tex.). Radiosynthesis of Composition ^{99m}Tc-SC-05-L-1 was achieved by adding ^{99m}Tc-pertechnetate (40-50 mCi) into the lyophilized residue of Compound SC-05-L-1 (5 mg) and tin (II) chloride (SnCl₂, 100 μg). The complexation of Compound SC-05-L-1 with ^{99m}Tc was carried out at pH 6.5.

Characterization of Composition ^{99m}Tc-SC-05-L-1

Radiochemical purity was determined by TLC (Waterman No. 1, Aldrich-Sigma, St. Louis, Mo.) eluted with acetone and saline. High-performance liquid chromatography (HPLC), equipped with a NaI detector and UV detector (280 nm), was performed on a PC HILIC Column (2.0 mm I.D.×150 mm, Agilent, Santa Clara, Calif.) eluted with acetonitrile/water (1:1 V/V) at a flow rate of 0.5 mL/min. Composition ^{99m}Tc-SC-05-L-1 was sat at 24 hr for extended shelf-life stability assays.

FIG. 2J and FIG. 2K show the radiochemical purity of Composition ^{99m}Tc-SC-05-L-1 synthesized in Example 4 of the invention in two different systems. Specifically, FIG. 2J shows the radiochemical purity of Composition ^{99m}Tc-SC-05-L-1 in an acetone system, and FIG. 2K shows the radiochemical purity of Composition ^{99m}Tc-SC-05-L-1 in a saline system. As shown in FIG. 2J and FIG. 2K, the radiochemical purity of Composition ^{99m}Tc-SC-05-L-1 was greater than 95% with Rf value 0.1.

FIG. 2L and FIG. 2M show the labeling efficiency of Composition ^{99m}Tc-SC-05-L-1 synthesized in Example 4 of the invention in two different systems. Specifically, Composition SC-05-L-1 (5 mg in 100 μL saline) was added 100 μg tin (II) chloride (in 100 μL H₂O) followed by 200 μL Na^{99m}TcO₄⁻ (˜5 mCi). As shown in FIG. 2L (280 nm channel) and FIG. 2M (radiostar channel), HPLC analysis of Composition ^{99m}Tc-SC-05-L-1 shows the retention time around 7 min.

FIG. 2N and FIG. 2O show the in vitro stability of Composition ^{99m}Tc-SC-05-L-1 synthesized in Example 4 of the invention in two different systems. Specifically, the in vitro stability of Composition ^{99m}Tc-SC-05-L-1 was measured after incubation at room temperature for 24 hr. As shown in FIG. 2N (280 nm channel) and FIG. 2O (radiostar channel), Composition ^{99m}Tc-SC-05-L-1 was stable in pH 6.5 after 24 hr.

### Example 5

Synthesis of Compound SC-05-N-1 and Composition ^{99m}Tc-SC-05-N-1

In this example, Compound SC-05-N-1 of the present invention was synthesized. The synthesis of Compounds 1-3 is similar to that of Compounds 1-3 described above, and is not repeated herein.

I. Synthesis of Compound 7

Di-tert-butyl 2,2′-(1,4,8, 1 1-tetraaza cyclotetradecane-1,8-diyl) diacetate (2,3-TADA-tBu) was first synthesized by a known method with 36% yield. The obtained 2,3-TADA-tBu was then conjugated to the epoxide analog of tamoxifen (compound 3 obtained above). More specifically, a solution of compound 3 (529 mg, 1.089 mmol) in ethanol (5 mL) was added slowly to a solution of 2,3-TADA-tBu (1870 mg, 4.363 mmol) in ethanol (5 mL). The reaction mixture was stirred under reflux overnight. The mixture was then cooled to room temperature and the solvent was removed under reduced pressure. The residue was purified by column chromatography to give compound 7 (637.8 mg, 0.697 mmol, 64%) as a white solid.

J. Synthesis of Compound SC-05-N-1

Compound 7 (226.5 mg, 0.248 mmol) and triethylsilane (0.2 mL, 1.238 mmol) were dissolved in dichloromethane (4 mL) and sat in ice-bath. Trifluoroacetic acid (2 mL) was slowly added. The reaction mixture was stirred at room temperature for overnight and monitored by TLC. The solution and excess reagent were removed under vacuum after the reaction was completed. The crude product was purified by reverse phase column chromatography with eluent from water to methanol to afford a white solid product compound SC-05-N-1.

K. Synthesis of Composition ^{99m}Tc-SC-05-N-1

Radiosynthesis of ^{99m}Tc-SC-05-N-1 was achieved by adding a required amount of ^{99m}Tc-pertechnetate into a vial containing SC-05-N1 (5 mg) and SnCl₂ (100 μg). Final pH of the preparation was 5.5-7.4. Radiochemical purity was determined by instant-TLC (Whatman 1001-150, Sigma-Aldrich, USA) eluting with saline and HPLC (PC HILIC column, 5 μm, 2.0 mm I.D.×150 mm, Milford, Mass.) eluting with water/acetonitrile/acetic acid (1/1/0.001) using flow rate of 1 mL/minute at UV 235 nm. The radiochemical purity of ^{99m}Tc-SC-05-N-1 was greater than 95%.

Characterization of Compound SC-05-N-1

NMR data was collected from 500 MHz Varian Inova NMR spectrometer (Palo Alto, Calif.) equipped with 5 mm PFG Triple ¹H-¹³C-¹⁵N probe, 5 mm PFG ¹H-¹⁹C-¹⁵N-³¹P switchable probe and 4 mm ¹H-¹³C Nano probe. HPLC data was collected from Waters 2695 Separations Module (Milford, Mass.) equipped with PC HILIC Column, (5 μm, 2.0 mm I.D.×150 mm).

FIG. 3A shows the ¹H-NMR spectrum of Compound SC-05-N-1 synthesized in Example 5 of the invention. FIG. 3B shows the ¹³C-NMR spectrum of Compound SC-05-N-1 synthesized in Example 5 of the invention. FIG. 3C shows the HPLC spectrum of Compound SC-05-N-1 synthesized in Example 5 of the invention. The structure of SC-05-N-1 was confirmed by ¹H-NMR and ¹³C-NMR, and the analysis results are presented in FIG. 3A and FIG. 3B respectively. As shown in FIG. 3C, HPLC analysis of Compound SC-05-N-1 (pH 5-7) using HILIC column shows the retention time around 6.4 min, with chemical purity of 100%.

### Example 6

### In Vitro Cellular Uptake Studies

Experiment 1

Compound SC-05-K-1 and Compound SC-05-L-1 (5 mg each) were dissolved in 0.3 mL water at pH 5-6. SnCL₂ (0.1 mg in 0.1 mL) was added (prepared from 10 mg tin (II) in 10 mL water), then Na^{99m}TcO₄ (5 mCi in 0.1 mL) was added. The total volume was diluted with water to 1 mL. The cell uptake for each well was 5 mg/5 mCi/1 mL (0.1 mg/0.1 mCi/20 uL/well). Each well contained 10 μg molecule. Multi-cell lines were used for cell uptake assays. A 96-well plate was used for MCF-7 ER (+) cell uptake studies. Each well contained 200,000 MCF-7 cells in 150 μL serum free RPMI. Composition ^{99m}Tc-SC-05-K-1 and Composition ^{99m}Tc-SC-05-L-1 were added to each well containing cells in the culture medium for different intervals (1-4 hr). To ascertain the cell uptake was via ER mediated process, Estradiol (10-100 times) was added to the MCF-7 cells. The cell uptake was expressed as percent of total dose.

FIG. 4A and FIG. 4B show the MCF-7 cell uptake and blocking studies of Composition ^{99m}Tc-SC-05-K-1 and Composition ^{99m}Tc-SC-05-L-1 synthesized in Example 2 and Example 4 of the invention. As shown in FIG. 4A and FIG. 4B, both Composition ^{99m}Tc-SC-05-K-1 and Composition ^{99m}Tc-SC-05-L-1 showed good cell uptake. Particularly, cell uptake was decreased (30-40%) after adding estradiol in Composition ^{99m}Tc-SC-05-L-1 as shown in FIG. 4A.

Experiment 2

A 6-well plate was used for OVCAR3 ER (+) and TOV-112D ER (−) cell uptake studies. Each well contained 100,000 cells in 150 μL serum free RPMI. Composition ^{99m}Tc-SC-05-K-1 and Composition ^{99m}Tc-SC-05-L-1 were added to each well containing cells in the culture medium for different intervals (0-2 hr). To ascertain OVCAR3 cell uptake of Composition ^{99m}Tc-SC-05-L-1 was via an ER mediated process, a blocking study was conducted. For blocking study, the amount of estrone used was 1 μg/well which was 1% of Composition ^{99m}TC-SC-05-L-1 dose (0.1 mg/0.1 mCi/20 μL/well). The well containing cells in the culture medium was incubated for different intervals (0-2 hr). Subsequently, cells are washed with ice-cold phosphate-buffered saline PBS twice and trypsinized with 0.5 mL of trypsin solution to detach tumor cells. Protein concertation assay was used to determine the proteins in each well. The cells were lysed in the lysis buffer containing proteinase inhibitors (Roche Diagnostic, Mannheim, Germany). The protein concentration in the cell lysate was quantified using Bradford Method as described by the manufacture (Bio-RAD, Hercules, Calif., USA). The Bradford dye was diluted in distilled water (1:4) and filtered through filter paper (number 1, Whatman no. 1, Advantec Co. Ltd., Tokyo). Bovine serum albumin at the concentration of 1000 μg/ml, 500 μg/ml, 250 μg/ml, 125 μg/ml, 62.5 μg/ml, 31.25 μg/ml were used to build a standard curve. Protein samples were diluted in lysis buffer at 1:9. Diluted protein samples or standard were mixed with Bradford dye in 96 well, then the absorbance at 595 nm was recorded. The radioactivity concentration in the cells and culture medium was measured with a gamma counter (Packard, Conn.) and expressed as cpm/g of cells and cpm/g medium. The protein mass-to-medium radioactivity concentration ratio was calculated and plotted over time.

FIG. 5A and FIG. 5B show the OVCAR3 cell and TOV-112D cell uptake studies of Composition ^{99m}Tc-SC-05-K-1 and Composition ^{99m}Tc-SC-05-L-1 synthesized in Example 2 and Example 4 of the invention. As shown in FIG. 5A and FIG. 5B, cell uptake studies with Composition ^{99m}Tc-SC-05-L-1 and Composition ^{99m}Tc-SC-05-K-1 indicated that Composition ^{99m}Tc-SC-05-L-1 had higher uptake in ER (+) OVCAR3 cells than ER (−) TOV-112D cells. Also, Composition ^{99m}Tc-SC-05-L-1 had higher cell/media ratios than Composition ^{99m}Tc-SC-05-K-1.

FIG. 6 shows the OVCAR3 cell and TOV-112D cell uptake and blocking studies of Composition ^{99m}Tc-SC-05-L-1 synthesized in Example 4 of the invention. As shown in FIG. 6, the OVCAR3 cell uptake of Composition ^{99m}Tc-SC-05-L-1 was blocked 80% by estrone indicating an ER mediated process occurred.

Experiment 3

Two epithelial ovarian cancer (EOC) ER (+) cell lines derived from poorly differentiated serous solid tumors (TOV-112D) and the matched ascites with malignant adenocarcinoma of the ovary (OVCAR3) were selected for biologic. evaluation. The ovarian tumor cell line OVCAR-3 was HER-2/ERα and ERβ strong positive whereas TOV-112D was ERα negative, and ERβ moderate positive. For cell uptake studies, the compound was soluble in 0.3 mL water (pH 5-6). SnCl₂ (0.1 mL, prepared from 10 mg tin (II) in 10 mL water) was added. NaTcO₄ (5 mCi in 0.1 mL) was added. The total volume was 1 mL after diluting with water. Each well contained 5 mg/5 mCi/1 mL (0.1 mg/0.1 mCi/20 uL/well). The cells were cultured in high-glucose Dulbecco's modified Eagle's medium (DMEM) supplemented with 100 U/mL penicillin and 100 μg/mL streptomycin in a humidified atmosphere of 5% CO₂ at 37° C. in 6-well plates until 60˜70% confluent (50,000 cells/well). After washing the monolayers three times with fresh media, the cells were incubated in fresh cell culture media containing ^{99m}Tc-SC-05-L-1 and ^{99m}Tc-SC-05-N-1 at 0.37 MBq/mL for 15, 30, 60 or 120 min. To ascertain the cell uptake was vis ER (+) systems, the accumulation of ^{99m}Tc-SC-05-L-1 in ovarian tumor cells were pre-treated with estrone (1 mg/well). The cells were then harvested by gentle scraping, pelleted by centrifugation at 3,500 rpm for 2 min. The cell pellets and 0.1 mL of the radioactive supernatants were weighed, and radioactivity was counted using a Packard 5500 gamma counter (Perkin-Elmer, Billerica, Mass., USA). Protein concertation in the cell lysate in each well was quantified using Bradford Method as described by the manufacture (Bio-RAD, Hercules, Calif., USA). Radioactivity was expressed as cpm/g cells and cpm/mL medium, respectively. Cell-to-medium radioactivity concentration ratios were calculated and plotted versus time to evaluate radiotracer accumulation kinetics.

As illustrated in FIG. 7A and FIG. 7B, in vitro cell uptake of ^{99m}Tc-SC-05-L-1 and ^{99m}Tc-SC-05-N-1 showed that both compounds had higher cell/media ratios in OVAC-3 than TOV-112d at 2 hr. Protein concentration in each well was 1 mg. Cell uptake of ^{99m}Tc-SC-05-L-1 was blocked 80% by estrone indicating an ER mediated process occurred. Though ^{99m}Tc-SC-05-L-1 had higher cell/media ratios than ^{99m}Tc-SC-05-N-1 in OVCA-3 ER (+) cells, the in vivo maximum tolerated doses (MTDs) in SC-05-L1 and SC-05-N1 were 50 ug and 100 ug, respectively. With physical amount at 10 ug, it was difficult to optimize radiolabeling efficiency. As such, ^{99m}Tc-SC-05-N-1 was further selected for in vivo imaging studies due to its less toxic and superior water solubility than ^{99m}Tc-SC-05-L-1. The compound SC-05-N-1 was selected for further imaging studies at the dosage of 50 ug which was safe below its MTD and with superior water solubility (see Example 7).

### Example 6

### In Vitro Anti-Cancer Studies

Experiment 4

Effect of Composition SC-05-L-1 and Composition SC-05-K-1 against lymphoma cells was assessed by using cell viability assays in representative mantle cell lines and diffuse large B-cell lymphoma (DLBCL) cell lines.

FIG. 8 shows the effect of Composition SC-05-L-1 and Composition SC-05-K-1 against lymphoma cells of the invention. As shown in FIG. 8, these cell lines were over-expressed with cannabinoid receptors.

Experiment 5

The cells were treated with increasing concentration of Compound SC-05-L-1 and Compound SC-05-K-1. Representative DLBCL cell lines sensitive or less sensitive to Compound SC-05-L-1 and Compound SC-05-K-1 were compared.

FIG. 9A and FIG. 9B show the in vitro anti-cancer studies of Compound SC-05-L-1 synthesized in Example 3 of the invention. FIG. 10A and FIG. 10B show the in vitro anti-cancer studies of Compound SC-05-K-1 and Compound SC-05-L-1 synthesized in Example 1 and Example 3 of the invention. As shown in FIG. 9A and FIG. 9B, the in vitro anti-cancer studies indicated that Compound SC-05-L-1 had dose-dependent manner against lymphoma cells. As shown in FIG. 10A and FIG. 10B, both Compound SC-05-L-1 and Compound SC-05-K-1 showed similar dose-dependent manner against lymphoma cells. However, Compound SC-05-L-1 is less toxic than Compound SC-05-K-1. In other words, the chelator cyclam is less toxic than chelator cyclen.

### Example 7: Animal Studies

All the experiments were carried out on 8-9-weeks-old male nude mice. The animals were housed with free access to water and maintained under controlled temperature (22±2° C.) and humidity conditions (55-65%) on a 12-h light/dark cycle. All the experiments conducted on animals were approved by the National Yang-Ming University Institutional Animal Care and Use Committee (IACUC No: 1050910).

Prior to animal imaging studies, the maximum tolerated dosage (MTD) of unlabeled SC-05-L1 and SC-05-N1 in normal healthy rodents were determined. The stock solution was prepared by using 5 mg compound in 5 mL water. The mice (n=3/dosage) were administered intravenously at 0.01 mL-0.1 mL (10-100 ug) and MTD was determined. For imaging studies, SC-05-N1 (5 mg) was soluble in 1 ml water (pH 5-6) and an aliquot of 0.1 mL (0.5 mg) was used. SnCL2 (0.1 mL, prepared from 10 mg tin (II) in 10 mL water) was added. NaTcO4 (2 mCi in 0.1 mL) was added. The total volume was 1 mL after diluting with water. The solution was filtered through 0.22 um filter and yielded 1.6 mCi. The final concentration was 0.5 mg/1.6 mCi/1 mL. Dosage for each mouse was 160 uCi (50 ug).

Animal model creation (Tumor cell xenografts):

The ovarian tumor cells (OVCAR-3 and TOV-112D) were cultured in flasks in DMEM/F-12 medium supplemented with 10% FBS and antibiotics at 37° C. in a humidified atmosphere with 5% CO2. Subcutaneous (s.c.) OVCAR-3 and TOV-112D tumor xenografts (10⁷ cells/type/mouse) were established in the opposite shoulder regions of athymic nu/nu mice (n=6 mice per pair of cell lines) to facilitate direct comparisons of accumulation between ^{99m}Tc-SC-05-N1 by SPECT/CT and ¹⁸F-FDG by PET/MR. When the tumors reached 5-8 mm in diameter, the tumor-bearing rodents were used for in vivo imaging studies.

Spect/Ct Imaging:

Small-animal SPECT/CT scans (n=6 per tumor pair) were performed using a SPECT/CT system (NanoScan; Mediso, Budapest, Hungary). Each tumor-bearing mouse was injected via tail vein with 37 MBq (1 mCi) of ^{99m}Tc-SC-05-N1. At 1 h, 2 h and 4 h after injection, the mice were anesthetized by inhalation of 2% isoflurane and imaged using the Nano-SPECT/CT camera. The SPECT and CT fusion images were obtained using the automatic fusion software (InterView Fusion; Mediso Medical Imaging Systems, Budapest, Hungary) and analyzed with PMOD 3.7 software (PMOD Technologies Ltd., Zurich, Switzerland). The biodistribution studies were conducted at the end of imaging studies. Computer-outlined tumor-to-muscle count density ratios and tumor uptake (% of injected dose) were determine.

Pet/MR Imaging:

The animals (n=6 per tumor pair) were anesthetized through passive inhalation of a mixture of isoflurane and oxygen (5% isoflurane for induction and 2% for maintenance) and then injected with [¹⁸F]FDG (19.4±1.8 MBq) via the tail vein. Static PET images were obtained using a small animal 7T PETMR Inline (Bruker, Germany) for 10 min, with the energy window set to 350-650 keV. T1 and T2 MRI of the brain were obtained to determine the anatomical structure. The MRI sequences included 0.5-mm thickness coronal T2 Turbo RARE high-resolution (TR=3455 ms, TE=36 ms, Matrix=256×256, Average=8, slice number=30). During imaging acquisition, animals were monitored for the depth of anesthesia, pulse and respiration constantly during the imaging procedure, and in the unlikely event that an animal regains consciousness the scanning would be immediately stopped and the animal removed (to be humanely euthanized and removed from the study). After imaging acquisition, the PET images were reconstructed through three-dimensional ordered-subset expectation maximization. Regional radioactivity concentration (KBq/cc) of ¹⁸F-FDG was estimated from the mean pixel values within volumes of interest (VOI) corresponding to MR imaging results. Image data were decay corrected to injection time. The radioactivity concentration (KBq/cc) of VOI was converted to standard uptake value (SUV) and the mean and standard error of the mean (SEM) of radiotracer accumulation in various tissues were calculated. The PET/MR Data were analyzed with PMOD 3.7 software (PMOD Technologies Ltd., Zurich, Switzerland).

FIG. 11 shows the transaxial and whole-body planar imaging of mice bearing OVAC-3 and TOV-112D tumors administered with ^{99m}Tc-SC-05-N-1 synthesized in Example 5 of the invention. FIG. 12 shows the transaxial images of mice bearing OVAC-3 and TOV-112D tumors in ¹⁸F-FDG group. FIG. 13A and FIG. 13B shows the biodistribution of mice bearing OVAC-3 and TOV-112D tumors administered with ^{99m}Tc-SC-05-N-1 synthesized in Example 5 of the invention.

As illustrated in FIG. 11 and FIG. 12, the OVCAR-3 and TOV-112D tumor-xenograft athymic nu/nu mouse model, ^{99m}Tc-SC-05-N-1 had better tumor-to-muscle delineation than ¹⁸F-FDG. Transaxial and whole-body planar images of ^{99m}Tc-SC-05-N1 showed OVAC-3 tumor had better uptake than TOV-112D. Transaxial images of mice bearing OVAC-3 and TOV-112D tumors in ¹⁸F-FDG group showed poor uptake. Furthermore, as illustrated in FIG. 13A and FIG. 13B, tissue distribution technique was used to determine tumor uptake and tumor/muscle count density ratios in OVAC-3 and TOV-112d-bearing mice administering ^{99m}Tc-SC-05-N-1 at 1-4 hrs. Biodistribution studies revealed that tumor uptake in OVAC-3 was higher than TOV-112d in the tumor-bearing rodents. Average tumor/muscle count density ratios at 1-4 hrs for OVCAR-3 and TOV-112d were 4-6 and 2-3, respectfully. These results indicated that ^{99m}Tc-SC-05-N-1 had higher tumor uptake than ¹⁸F-FDG in differential diagnosis of ovarian cancer, which is more promising as a chelator-tamoxifen conjugate for ER pathway-directed systems imaging.

In summary, the present invention provides the composition for cross talk between the estrogen receptors and the cannabinoid receptors. The hydroxy group is incorporated in the finished product. In the composition of the present invention, the protected chelator is used as to react the expoxylated receptor ligand to form the chelator-receptor ligand conjugate. The technology platform may exploit conjugating antagonists and agonists and seeing their effects in various forms of diseases. Also, the composition may be further prepared in pharmaceutical formulations and kits using the chemical procedures known to skilled artisans. In addition, the method of synthesizing the composition is also provided, and the synthesis method may obviate the need of adding protecting groups to the receptor ligand and increase process efficiency and purify of the final product. Besides, the composition of the present invention may be used for imaging or treating CBRs and ERs associated diseases.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A composition for cross talk between estrogen receptors and cannabinoid receptors, comprising a conjugate of a chelator and a receptor ligand, and a metal ion,
wherein the chelator comprises a nitrogen containing tetraazacyclic ring, and the nitrogen containing tetraazacyclic ring is a cyclam-carboxylic acid, or a cyclen-carboxylic acid;
the receptor ligand comprises non-steroidal tamoxifen, wherein in the conjugate, the non-steroidal tamoxifen is joined with the chelator by a —CH₂—CH₂—CH₂-O-CH₂-CHOH— group, and in the —CH₂—CH₂—CH₂—O—CH₂—CHOH— group, the left terminal —CH₂ is directly joined to an ethylene of a triphenylethylene group in the non-steroidal tamoxifen, and the right terminal —CHOH— is joined to the chelator side; and
wherein the metal ion is ^{99m}Tc ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³SM, ⁸⁹Sr, ⁵⁹Fe, ²¹²Bi, ²¹¹At, ⁴⁵Ti, Tc, Sn, Cu, In, Tl, Ga, As, Re, Ho, Y, Sm, Se, Sr, Bi, Fe, Mn, Lu, Co, Pt, Ca, Rh, Eu, Tb, or a combination thereof.

2. The composition according to claim 1, wherein the composition is a ^{99m}Tc-cyclam-tamoxifen analogue or a ^{99m}Tc-cyclen-tamoxifen analogue.

3. The composition according to claim 1, wherein the metal ion is ^{99m}Tc, and the conjugate comprises a compound of formula SC-05-N-1:

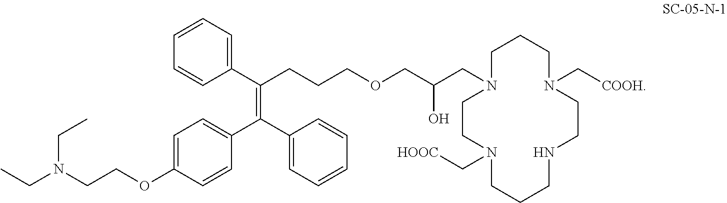 [SMILES: CCN(CC)CCOc1ccc(/C(=C(/CCCOCC(O)CN2CCCN(CC(=O)O)CCNCCCN(CC(=O)O)CC2)c2ccccc2)c2ccccc2)cc1]

4. A kit comprising the composition according to claim 1.

5. A method of synthesizing the composition according to claim 1, which comprises conjugating the receptor ligand to the chelator with an epoxide.

6. The method of synthesizing the composition according to claim 5, wherein the chelator is the nitrogen containing tetraazacyclic ring, and the receptor ligand is conjugated to the tetraazacyclic ring with the epoxide.

7. The method of synthesizing the composition according to claim 5, wherein the epoxide is attached to an aliphatic chain of the receptor ligand.

8. A treatment method for cancer, rheumatoid arthritis, osteoporosis, atherosclerosis, or endometrial tissue comprising administration of the composition according to claim 1.
